(19) United States
(12) Patent Application Publication
(10) Pub. No.: US 2022/0387358 A1
(43) Pub. Date: Dec. 8, 2022

(54) CERAMIDE ANALOG SACLAC MODULATES SPHINGOLIPID LEVELS AND MCL-1 SPLICING TO INDUCE APOPTOSIS IN ACUTE MYELOID LEUKEMIA

(71) Applicant: University of Virginia Patent Foundation, Charlottesville, VA (US)

(72) Inventors: Thomas P. Loughran, JR., Charlottesville, VA (US); David J. Feith, Palmyra, VA (US); Su-Fern Tan, Charlottesville, VA (US); Jennifer M. Pearson, Charlotte, NC (US); Mark Kester, Afton, VA (US); Tye Deering, Charlottesville, VA (US); Todd Fox, Charlottesville, VA (US); Helena Woodvine Snyder, Crozet, VA (US); Anuradha Illendula, Crozet, VA (US)

(21) Appl. No.: 17/775,213

(22) PCT Filed: Nov. 9, 2020
(86) PCT No.: PCT/US2020/059644

§ 371 Date: May 6, 2022

Related U.S. Application Data
(60) Provisional application No. 62/933,138, filed on Nov. 8, 2019.

(51) Int. Cl. A61K 31/164; A61K 31/635; A61K 31/706; A61K 9/127; A61K 9/00; A61P 35/02

(52) U.S. Cl. CPC A61K 31/164; A61K 31/635; A61K 31/706; A61K 9/1271; A61K 9/0019; A61P 35/02

(57) ABSTRACT

Provided are methods for treating a disease, disorder, or condition associated with an acid ceramidase (AC) biological activity. The methods include administering to a subject in need thereof a composition including an AC inhibitor and at least one additional active agent, such as a C6-ceramide nanoliposome (CNL); an inhibitor of a Bcl-2 family protein; a hypomethylating agent; an intensive chemotherapeutic agent such as cytarabine (AraC) and/or daunorubicin; a Hedgehog pathway inhibitor; a targeted agent, such as a FLT2 inhibitor or a EDH1/2 inhibitor; and/or an antibody drug conjugate that targets, for example, CD-33. The composition can include N-[(2S,3R)-1,3-dihydroxyoctadecan-2-yl]2-chloroacetamide (SACLAC) or a pharmaceutically acceptable salt thereof and at least one additional active agent. The disease, disorder, or condition associated with the AC biological activity can be a cancer, such as acute myeloid leukemia (AML).

### CROSS REFERENCE TO RELATED APPLICATION

The presently disclosed subject matter claims the benefit of U.S. Provisional Patent Application Ser. No. 62/933,138, filed Nov. 8, 2019, the disclosure of which incorporated herein by reference in its entirety.

### GOVERNMENT SUPPORT

This invention was made with government support under Grant Number P01CA171983 awarded by the National Institutes of Health. The government has certain rights in the invention.

### TECHNICAL FIELD

The presently disclosed subject matter relates generally to compositions and methods for treating and/or preventing a disease, disorder, or condition related to acid ceramidase (AC) biological activity. In some embodiments, the methods comprise administering to a subject in need thereof a composition comprising: (a) N-[(2S,3R)-1,3-dihydroxyoctadecan-2-yl]2-chloroacetamide (SACLAC) or another AC inhibitor and (b) at least one additional active agent, such as a C6-nanoliposome and/or venetoclax or another B-cell lymphoma 2 (Bcl-2) family protein inhibitor and/or cytarabine (AraC) and/or a hypomethylating agent. In some embodiments, the compositions and methods are related to treating acute myeloid leukemia (AML) in the subject.

### BACKGROUND

Acute myeloid leukemia (AML) is a malignancy of the blood and bone marrow, characterized by uncontrolled proliferation of immature myeloid blasts. The current prognosis for most patients is poor, with 5-year survival at only 27% (1). AML is more common in older populations, with a median age of 68 at diagnosis. Unfortunately, older patients have even worse prognoses, with only 7% of patients over the age of 65 surviving 5 years past diagnosis (1-3). Patients generally receive a combination of general chemotherapeutics cytarabine (7 days) and daunorubicin (3 days) by continuous infusion. While new therapies have been approved recently, there is little change in overall survival. Some targeted therapies are available for patients with genetic mutations such as FLT3 and JAK2 (4), but AML is an extremely heterogeneous disease where the most recurrent genetic abnormality only exists in about 30% of patients (5-7). Treatment with FLT3 inhibitor quizartinib achieved about 50% composite complete remission for FLT3-ITD-positive patients, but only about 5% of patients achieved complete remission (8).

There is an ongoing need for novel therapeutics in AML due to the limitations of current treatment options and poor patient survival (9,10), especially in the aging population that cannot tolerate intensive therapy (2,11). There are several hurdles involved in AML treatment, including therapy-related toxicity (12), genetic heterogeneity, de novo drug resistance and relapse (13). Identification of therapeutics that address one or more of these issues could greatly influence the future of AML treatment and improve patient prognosis.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method for treating a disease, disorder, or condition associated with an acid ceramidase (AC) biological activity, the method comprising administering to a subject in need thereof a composition comprising: (a) an AC inhibitor; and (b) at least one additional active agent, wherein the at least one additional active agent comprises at least one of a ceramide nanoliposome (CNL), an inhibitor of a Bcl-2 family protein, a hypomethylating agent, cytarabine (AraC), daunorubicin, a Hedgehog pathway inhibitor, a FMS-like tyrosine kinase 3 (FTL3) inhibitor, an isocitrate dehydrogenase 1 and/or 2 (IDH1/2) inhibitor, and an antibody conjugate, wherein the composition is administered via a route and in an amount effective for reducing the AC biological activity, thereby treating and/or preventing the disease, disorder, or condition associated with the AC biological activity.

In some embodiments, the disease, disorder, or condition associated with the AC biological activity is a cancer. In some embodiments, the cancer is acute myeloid leukemia (AML), optionally a venetoclax-resistant AML.

In some embodiments, the AC inhibitor is N-[(2S,3R)-1,3-dihydroxyoxtadecan-2-yl]-2-chloroacetamide (SACLAC) or a pharmaceutically acceptable salt thereof. In some embodiments, the at least one additional active agent comprises a CNL, optionally a C6-CNL.

In some embodiments, the at least one additional active agent comprises a Bcl-2 inhibitor and/or a Mcl-1 inhibitor. In some embodiments, the Bcl-2 inhibitor is venetoclax. In some embodiments, the at least one additional active agent further comprises AraC and/or a hypomethylating agent, optionally wherein the hypomethylating agent is azacytidine (AZA).

In some embodiments, the at least one additional active agent comprises AraC, optionally wherein the at least one additional active agent comprises AraC in combination with one or more of venetoclax, daunorubicin, and a Hedgehog pathway inhibitor, optionally glasdegib. In some embodiments, the at least one additional active agent comprises a hypomethylating agent, optionally wherein the hypomethylating agent is decitabine and/or AZA. In some embodiments, the at least one additional active agent comprises an antibody conjugate, optionally gemtuzumab ozogamicin, and/or a Hedgehog pathway inhibitor, optionally glasdegib. In some embodiments, the at least one additional active agent further comprises one or more of ivosidenib, enasidenib, midostaurin, gilteritinib, a histone deacetylase (HDAC) inhibitor, an epigenetic regulator, and a histone demethylase inhibitor.

In some embodiments, the presently dislcosed subject matter provides a method for treating AML, the method comprising administering to a subject in need thereof a composition comprising (a) an AC inhibitor; and (b) at least one additional active agent, wherein the at least one additional active agent comprises at least one of a CNL, an inhibitor of a Bcl-2 family protein, a hypomethylating agent, cytarabine (AraC), daunorubicin, a Hedgehog pathway inhibitor, a FTL3 inhibitor, an IDH1/2 inhibitor, and an antibody conjugate, wherein the composition is administered via a route and in an amount effective for treating the AML. In some embodiments, the AC inhibitor is SACLAC or a pharmaceutically acceptable salt thereof.

In some embodiments, the at least one additional active agent comprises a CNL, optionally a C6-CNL. In some embodiments, the at least one additional active agent comprises a Bcl-2 inhibitor. In some embodiments, the Bcl-2 inhibitor is venetoclax. In some embodiments, the at least one additional active agent comprises a Mcl-1 inhibitor. In some embodiments, the at least one additional active agent further comprises AraC and/or a hypomethylating agent, further optionally wherein the hypomethylating agent is AZA.

In some embodiments, the at least one additional active agent comprises AraC, optionally wherein the at least one additional active agent comprises AraC in combination with one or more of venetoclax, daunorubicin, and a Hedgehog pathway inhibitor, optionally glasdegib. In some embodiments, the at least one additional active agent comprises a hypomethylating agent, optionally wherein the hypomethylating agent is decitabine and/or AZA. In some embodiments, the at least one additional active agent comprises an antibody conjugate, optionally gemtuzumab ozogamicin, and/or a Hedgehog pathway inhibitor, optionally glasdegib. In some embodiments, the at least one additional active agent further comprises one or more of ivosidenib, enasidenib, midostaurin, gilteritinib, a HDAC inhibitor, an epigenetic regulator, and a histone demethylase inhibitor. In some embodiments, the AML, is a venetoclax-resistant AML

In some embodiments, the AC inhibitor and/or the one or more active agent is encapsulated in a polymer nanoparticle, wherein the polymer nanoparticle comprises a biodegradable and biocompatible polymer, optionally wherein the biodegradable and biocompatible polymer is selected from a polyethylene glycol-poly(lactic-co-glycolic acid) (PEG-PLGA) copolymer and a polyethylene glycol-poly(d,l-lactic acid) (PEG-PDLLA) diblock copolymer. In some embodiments, the subject is a mammalian subject, optionally a human subject.

In some embodiments, the presently disclosed subject matter provides a composition for use in treating a disease, disorder, or condition associated with an AC biological activity, the composition comprising (i) an AC inhibitor, optionally SACLAC or a pharmaceutically acceptable salt thereof, and (ii) at least one additional active agent, wherein the at least one additional active agent comprises at least one of a CNL, an inhibitor of a Bcl-2 family protein, a hypomethylating agent, cytarabine (AraC), daunorubicin, a Hedgehog pathway inhibitor, a FTL3 inhibitor, an IDH1/2 inhibitor, and an antibody conjugate, in an amount effective for reducing the AC biological activity in a subject.

In some embodiments, the presently disclosed subject matter provides a composition for use in treating AML, the composition comprising (i) an AC inhibitor, optionally SACLAC or a pharmaceutically acceptable salt thereof and (ii) at least one additional active agent, wherein the at least one additional active agent comprises at least one of a CNL, an inhibitor of a Bcl-2 family protein, a hypomethylating agent, cytarabine (AraC), daunorubicin, a Hedgehog pathway inhibitor, a FTL3 inhibitor, an IDH1/2 inhibitor, in an amount effective for treating AML in a subject.

In some embodiments, the at least one additional active agent comprises at least one of a Bcl-2 inhibitor, optionally venetoclax, a CNL, optionally a C6-ceramide nanoliposome, and a hypomethylating agent, optionally decitabine and/or AZA. In some embodiments, the at least one additional active agent comprises ventoclax, AraC, and/or AZA. In some embodiments, the composition comprises SACLAC, AraC, and venetoclax.

In some embodiments, the at least one additional active agent comprises daunorubicin, glasdegib, and/or gemtuzumab ozogamicin. In some embodiments, the composition further comprises one or more of ivosidenib, enasidenib, midostaurin, gilteritinib, a histone deacetylase (HDAC) inhibitor, an epigenetic regulator, and a histone demethylase inhibitor.

In some embodiments, the AC inhibitor and/or one or more of the one or more additional active agents are encapsulated in a polymer nanoparticle, wherein the polymer nanoparticle comprises a biodegradable and biocompatible polymer, optionally wherein the biodegradable and biocompatible polymer is selected from a PEG-PLGA copolymer and a PEG-PDLLA diblock copolymer.

In some embodiments, the presently disclosed subject matter provides a composition comprising an AC inhibitor, optionally SACLAC, encapsulated in a CNL or a polymer nanoparticle, wherein the polymer nanoparticle comprises a biodegradable and biocompatible polymer selected from a PEG-PLGA copolymer and a PEG-PDLLA diblock copolymer.

Accordingly, it is an object of the presently disclosed subject matter to provide compositions and methods for treating and/or preventing a disease, disorder, or condition associated with acid ceramidase biological activity, such as AML.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIGS. 1A-1D: SACLAC inhibits AC and shifts lipid levels toward a pro-death phenotype. FIG. 1A: SACLAC is an a-chloroamide ceramide analog. FIG. 1B: Acid ceramidase activity levels were measured by fluorogenic substrate conversion to determine AC inhibition after SACLAC (2.5 μM) treatment of HL-60/VCR, THP-1 and OCI-AML2 human AML cell lines for 24 hours. This experiment was repeated two to three times per cell line, and a representative experiment is shown. FIGS. 1C and 1D show sphingosine 1-phosphate and total ceramide levels, respectively, measured by mass spectrometry and normalized to total protein to determine sphingolipid changes in response to SACLAC (2.5 μM) treatment of human AML cell lines for 24 hours. This experiment contained five biological replicates and multiple doses with similar response. Statistical analysis was done using two-tailed unpaired t-test to compare DMSO and treatment. Lipid data was corrected for multiple testing using the Holm-Sidak method. *p<0.05, **p<0.01, ***p<0.001 relative to DMSO control. N.D. indicates values were below the limit of detection.

FIGS. 2A-2C: SACLAC reduces cell viability and colony formation. FIG. 2A: Viability of three human AML cell lines was measured by MTS assay after 24-hour treatment with the indicated dose of SACLAC. FIG. 2B: Colony formation assay measured clonogenic potential in a panel of six primary AML patient samples with increasing doses of SACLAC. This experiment contained six distinct patient samples as biological replicates. FIG. 2C: The concentration of SACLAC required to achieve a 50% reduction in cell viability (EC₅₀) in 30 different human AML cell lines was measured by MTS at 24 and 48 hours. Each dot represents one MTS assay (n=2-6). Global p-value (ANOVA) is presented on the graph. Asterisks denote pairwise comparison from Dunnett's Test comparing each dose to DMSO control. ** p<0.01, ***p<0.001.

FIGS. 3A-3E: SACLAC induces apoptosis and loss of mitochondrial membrane potential. OCI-AML2 cells were treated with DMSO or SACLAC (5 μM) and evaluated for (FIG. 3A) apoptosis and (FIG. 3B) mitochondrial membrane depolarization at the indicated time. FIG. 3C: Caspase 3/7 activation was determined in OCI-AML2 cells treated with increasing doses of SACLAC for 24 hours. Dose-dependent apoptosis was measured in (FIG. 3D) human AML cell lines and (FIG. 3E) primary patient samples with 48-hour SACLAC treatment (μM). Studies of patient samples were performed once with a panel of seven distinct patient samples and the line represents the group mean. Global p-value (ANOVA) is presented on the graph. Asterisks denote pairwise comparison from Dunnett's Test. Dose responses are compared to DMSO and time courses are compared to 0-hour time point. * p<0.05, **p<0.01, ***p<0.001.

FIGS. 4A-4F: SACLAC reduces expression of SF3B1 and increases pro-apoptotic Mcl-1S. FIG. 4A: OCI-AML2 cells were treated with the indicated dose of SACLAC for 24 hours, then SF3B1, Mcl-1S and Mcl-1L protein expression was measured by western blot. SF3B1 (FIG. 4B) and Mcl-1S/L ratio (FIG. 4C) were quantified relative to β-actin loading control and compared to DMSO vehicle control. FIG. 4D: Alternative splicing of Mcl-1 in OCI-AML2 cells treated for 24 hours with SACLAC (5 μM) was clearly distinguished from proteolytic cleavage of Mcl-1 induced by ABT-737 (75 nM) as a positive control. Appearance of the Mcl-1 cleavage product, but not Mcl-1S, was blocked by caspase inhibitor Z-VAD-FMK (50 μM). FIG. 4E: SF3B1 involvement in Mcl-1 splicing was confirmed using 5 (+), 10 (++) or 20 nM (+++) spliceostatin A (SSA) to treat OCI-AML2 cells for 6 hours. This experiment was repeated twice. FIG. 4F: MCL-1S transcript levels after 6-hour treatment with 5 μM SACLAC were measured using RT-qPCR. This experiment was repeated twice. Global p-value (ANOVA) is presented on the graph. Asterisks denote pairwise comparison from Dunnett's Test comparing each dose to DMSO control. RT-qPCR analysis utilized two-tailed unpaired t-test to compare DMSO and treatment. * p<0.05, **p<0.01, ***p<0.001. SAC, SACLAC; VAD, Z-VAD-FMK; ABT, ABT-737; SSA, spliceostatin A.

FIGS. 5A-5F: C16 ceramide treatment and AC knockdown reduce SF3B1 and alter Mcl-1S to L ratio. FIG. 5A: SF3B1, Mcl-1L and Mcl-1S protein expression was measured by western blot after treatment with 20 μM C16 ceramide for 48 hours in OCI-AML2 human AML cells. SF3B1 (FIG. 5B) and Mcl-1S/L ratio (FIG. 5C) were quantified relative to β-actin loading control and compared to DMSO vehicle control. FIG. 5D: KG1a cells were electroporated with siRNA (50 nM) targeting AC and harvested 48 hours later. Knockdown was confirmed and changes in protein levels were evaluated using western blotting. Change in (FIG. 5E) SF3B1 level and (FIG. 5F) Mcl-1S/L ratio were quantified relative to scrambled siRNA as the control. Statistical analysis was done using two-tailed unpaired t-test to compare control (vehicle or scrambled siRNA) and treatment (C16 or targeting siRNA). *p<0.05, **p<0.01.

FIGS. 6A-6F: Altering expression of Mcl-1 and SF3B1 attenuates induction of apoptosis in SACLAC-treated cells. FIG. 6A: KG1a cells were electroporated with siRNA (100 nM) targeting the MCL-1S transcript. After 24 hours, cells were treated with 10 μM SACLAC and analyzed by western blot for Mcl-1S protein expression 48 hours later. Mcl-1L (FIG. 6B) and SF3B1 (FIG. 6C) were overexpressed in KG1a cells by electroporating with 15 μg plasmid cDNA and then treated with 7.5 μM SACLAC, respectively, 24 hours later. Protein quantification is shown relative to DMSO control following normalization to β-actin. Mcl-1L is expressed as a Mcl-1/EGFP fusion protein in B; therefore, no quantitation is provided in empty vector lanes. The ability of (FIG. 6D) Mcl-1S knockdown, (FIG. 6D) Mcl-1L overexpression and (FIG. 6F) SF3B1 overexpression to rescue cells from SACLAC mediated cell death was measured by comparing apoptosis induction over baseline (apoptosis in DMSO control group) for each condition. Overexpression experiments were repeated twice with two doses showing similar results. Statistical analysis utilized two-tailed unpaired t-test to compare control (scrambled siRNA or empty vector) and treatment (targeting siRNA or cDNA). ** p<0.01, ***p<0.001. Lines in blots indicate non-adjacent lanes on a single blot at the same exposure.

FIGS. 7A-7F: SACLAC reduces leukemic burden in NSG mouse models of AML. FIG. 7A: NOD-scid IL2Rgamma^{null} (NSG) mice (n=3 per group) were injected with 2.5×10⁶ human MV4-11 cells labeled with YFP-Luc, and engraftment was confirmed by bioluminescence imaging after two weeks. Each animal received five tail-vein injections per week of SACLAC (5 mg/kg SACLAC in 45% w/v 2-hydroxypropyl-β-cyclodextrin in PBS) or vehicle control. After 18 injections, leukemic burden was measured in blood by flow cytometry staining for (FIG. 7B) hCD45 and (FIG. 7C) YFP markers to identify MV4-11 cells. FIG. 7D: NSG mice (n=5 per group) were injected with 1×10⁴ human U937 cells labeled with tdTomato-Luc, and engraftment was confirmed by bioluminescence imaging after one week. Each animal received five tail-vein injections per week of SACLAC (5 mg/kg SACLAC in 45% w/v 2-hydroxypropyl-β-cyclodextrin in PBS) or vehicle control. After 15 injections, leukemic burden was measured by flow cytometry staining for (FIG. 7E) hCD45 and (FIG. 7F) tdTomato markers to identify U937 cells in bone marrow. Statistical analysis utilized two-tailed unpaired t-test to compare vehicle and SACLAC treatment. * p<0.05.

FIGS. 8A-8C: SACLAC reduces AC activity at lower doses than LCL204. A fluorogenic substrate was used to measure AC activity after treatment with DMSO vehicle, LCL204 (5 or 10 μM), or SACLAC (1.25 or 2.5 μM) in (FIG. 8A) HL-60/VCR, (FIG. 8B) THP-1 and (FIG. 8C) OCI-AML2 cell lines. These experiments were repeated twice with equivalent results, and one representative experiment is shown. To demonstrate that SACLAC is more potent than LCL204, comparisons were done on 4X concentrated LCL204 versus 1× SACLAC (5 vs. 1.25 and 10 vs. 2.5 μM) using two-tailed unpaired t-test. ** p <0.01,***p<0.001.

FIGS. 9A-9C: SACLAC treatment increases production of multiple ceramide species in human AML cell lines. Mass spectrometry was used to measure ceramide content after treatment with DMSO vehicle or SACLAC (2.5 μM) for 24 hours in (FIG. 9A) HL-60/VCR, (FIG. 9B) THP-1 and (FIG. 9C) OCI-AML2 cell lines. The y-axis shown is logio to accommodate all species on an unbroken axis. Each bar represents the mean±SEM of 5 independent biological replicates from a single experiment. Similar response was observed at other doses (1.25 and 5 μM) of SACLAC. *p<0.05, **p<0.01, ***p<0.001. # indicates that bar value falls below lower axis limit. Comparison of DMSO versus SACLAC for each ceramide species was evaluated using two-tailed unpaired t-test. p-values were corrected for multiple testing using the Holm-Sidak method.

FIG. 10: SACLAC is more toxic to AML cells than normal cells. Pooled EC₅₀ values are depicted for 29 AML cell lines (AML, n=29) and four normal CD34+ samples and six normal PBMC samples (Normal, n=10). Comparison of AML cell lines versus normal cells was done using two-tailed unpaired t-test. * p<0.05.

FIGS. 11A-11E: SACLAC mechanism of action is consistent in multiple human AML cell lines. THP-1 cells were treated with DMSO or SACLAC (10 μM) for 0 to 24 hours and evaluated for (FIG. 11A) apoptosis and (FIG. 11B) mitochondrial membrane depolarization. Data presented is from two independent experiments with equivalent results. FIG. 11C: THP-1, HL-60/VCR, and KG1a cells were treated with DMSO (−) or 20 μM SACLAC (+) for 48 hours and assayed for protein levels via western blotting. A representative blot is shown with fold change relative to DMSO and normalized to β-actin listed below the blots for SF3B1, Mcl-1L (top) and S (bottom) variants. SF3B1 level (FIG. 11D) and Mcl-1S/L ratio (FIG. 11E) from three independent experiments completed as in C were quantified by normalizing to β-actin loading and compared to DMSO control. * p<0.05, **p<0.01, ***p<0.001. FIGS. 11A and 11B, the global p-value (ANOVA) is presented along with asterisks denoting significance of each time point of SACLAC treatment relative to time 0 using a Dunnett's test. Values in FIG. 11C show quantification of Mcl-1L (upper) and Mcl-1S (lower) relative to DMSO (set to 1). For FIGS. 11D and 11E, SACLAC treatment was compared to DMSO for each cell line and analyzed using two-tailed unpaired t-test.

FIGS. 12A-12B: Exogenous ceramides induce apoptosis in human AML cell lines. FIG. 12A: HL-60/VCR, THP-1, and OCI-AML2 human AML cell lines were treated with long-chain ceramide mixture (100 μg/ml) and assayed for apoptosis at 48 hours. FIG. 12B: OCI-AML2 cells were treated with DMSO or C16 ceramide (20 μM) for 48 hours and assayed for apoptosis. Comparison of vehicle versus ceramide was done using two-tailed unpaired t-test. *** p<0.001. Bars represent mean±SEM of three independent experiments.

FIG. 13. Baseline apoptosis varies based on type of electroporated content. Cells were electroporated with control (non-targeting siRNA or empty vector) or targeted agent (targeting siRNA or targeting cDNA expression vector) and then treated with DMSO vehicle control to determine baseline toxicity with each manipulation.

FIG. 14. Proposed model of SACLAC mechanism of action. SACLAC treatment inhibits AC which leads to increased ceramide and decreased SF3B1. As ceramide accumulates, alternative splicing of Mcl-1 results in Mcl-1S accumulation that facilitates mitochondrial membrane depolarization. With loss of mitochondrial membrane potential (ΔΨm), pro-apoptotic signals are released to activate caspases and induce apoptosis.

FIGS. 15A-15B: IV administration of SACLAC improves delivery and lipids are shifted in mice. SACLAC was administered to Swiss Webster mice intraperitoneally in DMSO (40 mg/kg) or intravenously in β-HPCD (5 mg/kg). Animals were killed at the indicated time points and concentration of (FIG. 15A) SACLAC in blood or (FIG. 15B) sphingosine in the liver was measured by mass spectrometry. Values represent mean±SEM from 3 mice per time point. Comparison between DMSO and β-HPCD concentration was evaluated using two-tailed unpaired t-test. Change in sphingosine was evaluated using ANOVA followed by Dunnett's test comparing each time point to 0 hours. * p<0.05, **p<0.01.

FIG. 16: Delivery of SACLAC in nanoliposome or 2-hydroxypropyl-β-cyclodextran (β-HPCD). Mouse serum SACLAC levels following delivery at the indicated dose, formulation and route.

FIG. 17: SACLAC synergy with C6-ceramide nanoliposome (CNL). Loss of viability in THP-1 hAML cells treated with SACLAC or SACLAC+CNL (10 μM; 24 h). Inset shows dose-dependent AC induction with CNL treatment (24 h).

FIGS. 18A-18B: SACLAC synergy with venetoclax. SACLAC plus venetoclax treatment induced synergistic killing in (FIG. 18A) venetoclax-sensitive MV4-11 cells (24 h) and (FIG. 18B) venetoclax-resistant MM-6 cells (48 h).

FIG. 19: SACLAC synergy with AraC/venetoclax. SACLAC treatment induced synergistic killing when combined with the AraC/venetoclax combination, including in cells that overexpress mutant DNMT3A, NPM1 and FLT3.

FIGS. 20A and 20B: SACLAC synergy with hypomethylating agents. SACLAC treatment induced synergistic killing in drug resistant HL-60-VCR cells when combined with (FIG. 20A) azacytidine (48 h) and (FIG. 20B) decitabine (72 h).

### DETAILED DESCRIPTION

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art.

References to techniques employed herein are intended to refer to the techniques as commonly understood in the art, including variations on those techniques or substitutions of equivalent techniques that would be apparent to one of skill in the art. In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a therapeutic agent” refers to one or more therapeutic agents, e.g., one or more of the same of different therapeutic agents. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of time, concentration percent inhibition, percent viability, amounts of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “acid ceramidase” is understood to mean an amidase enzyme that catalyzes the conversion of ceramide or ceramide-based substrates to their respective sphingosine or sphingosine-containing analogs via a deacylation reaction.

The term “acid ceramidase inhibitor” is understood to mean a compound that preferentially reduces the activity of an acid ceramidase enzyme relative to other mammalian enzymes, for example, other enzymes present in lysosomes of mammalian cells.

The terms “additional therapeutically active compound” “additional active agent and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A: T and G: C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer. In some embodiments, the disease is leukemia, which in some embodiments is Acute Myeloid Leukemia (AML).

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions. As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.)

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented. The term “treating” refers any effect, e.g., lessening, reducing, modulating, ameliorating, reversing or eliminating, that results in the improvement of the condition, disease, disorder, and the like, or ameliorating a symptom thereof.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. General Considerations

An emerging area of study in cancer therapy involves manipulating sphingolipid metabolism in cancer cells to control cell fate (14). While sphingolipids are generally perceived as structural components of cellular membranes, there are two key bioactive sphingolipids at the center of this pathway. Ceramide is a known second messenger in cell death while sphingosine 1-phosphate (S1P) is pro-survival (15). Enzymes that mediate the conversion of ceramide to S1P are tightly regulated to maintain the balance between the integrity of healthy cells and the destruction of damaged cells. However, dysregulation of the enzymes regulating this pathway can contribute to many diseases including cancer (14,16,17).

Acid ceramidase (AC, also referred to as N-acylsphingosine amidohydrolase 1 (ASAH1) plays an important role in balancing these two lipids (17). AC is part of a family of lipid hydrolases that cleave ceramide to form sphingosine, which can be subsequently phosphorylated to produce S1P. AC is upregulated in several cancers (18-21). For example, AC is the highest expressed and most upregulated ceramidase in AML and mediates survival of AML cells (22). Elevated AC activity contributes to increased P-glycoprotein (P-gp) expression and a drug resistance phenotype in AML (23). AC upregulation was observed in most AML patient samples, indicating that AC is a promising therapeutic target that may be applicable to a large percentage of AML patients.

AC inhibition with LCL204 coincides with a loss of pro-survival Mcl-1 protein, leading to apoptosis (22). This finding is of interest given the role of Bcl-2 family members in AML pathogenesis and response to therapy (24). For example, upregulation of Mcl-1 is an established mechanism of resistance to the promising Bcl-2 inhibitor venetoclax, which is currently approved for use in AML in elderly patients in combination with hypomethylating agents or low dose cytarabine (25-27). The full-length pro-survival Mcl-1 protein can also be referred to as Mcl-1L. However, Mcl-1 function can be altered through alternative mRNA splicing (28,29). When splicing is disrupted, exclusion of exon 2 results in formation of Mcl-1S. This step is regulated by SF3B1, a component of spliceosome assembly (28). This alternative splicing event can result in a change of function—Mcl-1L is pro-survival while Mcl-1S is pro-apoptotic (30). Mcl-1S retains the functional BH3 domain, which is involved in mitochondria-mediated induction of apoptosis (30,31). Previous studies have demonstrated that modulation of mRNA splicing can induce apoptosis of cancer cells (28,32).

According to one aspect of the presently disclosed subject matter, AC inhibition is utilized to increase ceramide levels and induce selective toxicity of AML cells. As described further in the Examples, an exemplary AC inhibitor, SACLAC, was studied to determine its efficacy and mechanism of action in AML cell lines, patient samples and xenograft models. By targeting AC, a common biochemical dependence in AML cells can be exploited and can provide a therapeutic approach that is broadly applicable to the diverse population of AML patients.

More particularly, as described further in the Examples, an exemplary AC inhibitor, SACLAC, is characterized. SACLAC significantly reduces viability of AML cells with an EC₅₀ of approximately 3 μM across 30 human AML cell lines. Treatment of AML cell lines with SACLAC effectively blocks AC activity, induces a decrease of sphingosine 1-phosphate and a 2.5-fold increase in total ceramide levels. Mechanistically, SACLAC treatment leads to an unexpected reduction in levels of splicing factor SF3B1 and alternative Mcl-1 mRNA splicing in multiple human AML cell lines. This increases the levels of pro-apoptotic Mcl-1S and contributed to SACLAC-induced apoptosis in AML cells. The apoptotic effects of SACLAC are attenuated by SF3B1 or Mcl-1 overexpression, as well as by selective knockdown of Mcl-1S. Furthermore, AC knockdown and exogenous supplementation with C16-ceramide induces similar changes in SF3B1 and Mcl-1S/L ratio. In addition, SACLAC treatment leads to a 37 to 75% reduction in leukemic burden in two human AML xenograft mouse models. These data emphasize AC as a therapeutic target in AML and define SACLAC as a potent inhibitor. Furthermore, SACLAC delivery can be enhanced by using nanoliposome or cyclodextrin-based formulations and SACLAC shows synergistic effects with C6-ceramide nanoliposomes (CNL), venetoclax, AraC/venetoclax, and hypomethylating agents, such as azacytidine and decitabine.

### III. Exemplary Methods and Compositions

In some embodiments, the presently disclosed subject matter provides a method for treating a disease, disorder, or condition associated with an acid ceramidase (AC) biological activity. In some embodiments, the method comprises administering to a subject in need thereof a composition comprising a combination of an AC inhibitor and at least one additional active agent. In some embodiments, the method comprises administering to a subject in need thereof a composition comprising: (a) an AC inhibitor; and (b) at least one (e.g., one, two, three, four, five, or more) additional active agents selected from the group including, but not limited to: a ceramide nanoliposome (CNL); an inhibitor of a B-cell lymphoma 2 (Bcl-2) family protein (e.g., a Bcl-2 protein, a myeloid cell leukemia 1 (Mcl-1) protein, or a B-cell lymphoma-extra-large (Bcl-XL) protein); a hypomethylating agent; cytarabine (AraC); daunorubicin; a Hedgehog pathway inhibitor; a targeted agent, such as a FMS-like tyrosine kinase 3 (FTL3) inhibitor or an isocitrate dehydrogenase 1 and/or 2 (IDH1/2) inhibitor; and an antibody conjugate (e.g., an antibody-drug conjugate where the antibody is targeted to a receptor found on a cell surface, e.g., CD-33, and where the drug is a small molecule that has a therapeutic effect, e.g., for treating cancer); wherein the composition is administered via a route and in an amount effective for reducing the AC biological activity, thereby treating and/or preventing the disease, disorder, or condition associated with the AC biological activity.

Diseases, disorders, and conditions associated with AC biological activity treatable according to the presently disclosed methods include diseases, disorders and conditions related to cell proliferation, cell attachment, cell migration, granulation tissue development, primary and metastatic neoplastic diseases, diseases and disorders related to inborn errors in lipid metabolism, inflammation, cardiovascular disease, stroke, ischemia or atherosclerosis. Diseases and disorders involving cell overproliferation that can be treated include, but are not limited to cancers, premalignant conditions (e.g., hyperplasia, metaplasia, dysplasia), benign tumors, hyperproliferative disorders, and benign dysproliferative disorders. In some embodiments, the diseases, disorders or conditions treatable according to the presently disclosed subject matter include cancer, cancer metastasis, atherosclerosis, stenosis, inflammation, asthma, Alzheimer's disease and atopic dermatitis. Cancers and related disorders that can be treated or prevented by methods and compositions of the present invention include but are not limited to the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, large granular lymphocyte leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's acroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipidus; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/ or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In some embodiments, the presently disclosed methods and compositions are for treating cancer. In some embodiments, the cancer is selected from a leukemia, a breast cancer, a colon cancer, an ovarian cancer, a lung cancer, a prostate cancer, and a melanoma. In some embodiments, the cancer is acute myeloid leukemia (AML). In some embodiments, the AML is a drug resistant or sensitive AML, such as a venetoclax-resistant AML or a venetoclax-sensitive AML. In some embodiments, the AML is a venetoclax-resistant AML. In some embodiments, the AML is a newly diagnosed AML. In some embodiments, the AML is a relapsed/recurring AML (RR-AML).

A variety of AC inhibitors have been reported. Small molecule AC inhibitors were described, for example, in a 2014 review (64) AC inhibitors suitable for use according to the presently disclosed subject matter include, but are not limited to, ceramide analogs, such as (1R, 2R)-2-N-(tetradecanoylamino)-1-(4′-nitrophenyl)-1,3-propandiol (also known as B13), (1R, 2R)-2-N-(tetradecylamino)-1-(4′-nitrophenyl)-1,3-propandiol (also known LCL204), (1R,2R)-1-(4-nitrophenyl)-2-tetradecanamidoproopane-1,3-diyl bis(2-(dimethylamino)acetate dihydro-chloride (also known as LCL521) (65), E-tb, N-oleoylethanolamine-1S,2R—N-myristoylaminophenylpropanol-1 (D-e-Mapp), N-[(2S,3R)-1,3-dihydroxyoctadecan-2-yl]2-chloroacetamide (SACLAC), and 2-bromo-N-((2S,2R)-1,3-dihydroxyactadecan-2-yl)acetamide (SABRAC); 5-chloro-3-[hexylamino)carbonyl]-3,6-dihydro-2,6-dioxo-1(2H)-pyrimidine carboxylic acid, 2-methylpropyl ester (also known as ARN14988) (66); quinolinones, such as 3-[(2E)-3-(4-methoxyphenyl)-1-oxo-2-propen-1-yl]-6-methyl-4-phenyl-2(1H)-quinolinone (also known as Ceranib 1) and 3-[3-(4-methoxyphenyl)-1-oxo-2-propen-1-yl]-4-phenyl-2(1H)-quinolinone (also known as Ceranib 2), urea-substituted benzimidazoles (67) benzoxazolone carboxamides (68); and uracil analogs, such as 5-fluorouracil analogs, e.g., 1-hexylcarbamoyl-5-fluorouracil (also known as Carmofur). In some embodiments, the AC inhibitor is N-[(2S,3R)-1,3-dihydroxyoxtadecan-2-yl]-2-chloroacetamide (SACLAC) or a pharmaceutically acceptable salt thereof.

In some embodiments, the at least one additional active agent comprises a CNL. Thus, in some embodiments, the method comprises administering a CNL e.g., that is associated with the AC inhibitor and/or one or more of the additional active agents, such as decitibine, azacitadine, AraC, venetoclax, or any combination or subcombination thereof. The preparation and use of CNLs is described, for example, in U.S. Pat. No. 8,747,891 and U.S. Patent Application Publication Nos. 2020/0170970, 2020/0268665, each of which is incorporated herein by reference in its entirety. CNLs can comprise lipid bilayers comprising one or more C2-C24 ceramides. In some embodiments, the CNL comprises a C6 ceramide (i.e., a C6-CNL). The CNL can further include one or more neutral, cationic or anionic lipid. In some embodiments, the CNL comprises a poly(ethylene glycol) (PEG)-modified lipid.

Bcl-2 family protein inhibitors include, but are not limited to, inhibitors of Bcl-2 protein, inhibitors of Mcl-1 protein, and inhibitors of Bcl-XL protein. In some embodiments, the Bcl-2 family protein inhibitor is an inhibitor of Bcl-2. In some embodiments, the at least one additional active agent is a Bcl-2 protein inhibitor (i.e., a Bcl-2 inhibitor) and/or a Mcl-1 inhibitor.

In some embodiments, the at least one additional active agent is a Bcl-2 inhibitor. Bcl-2 inhibitors include, but are not limited to, venetoclax (ABT-199), navitoclax, 4-[4-[[2-(4-chlorophenyl)phenyl]methyl]piperazin-l-yl]-N-[4-[[(2R)-4-(dimethylamino)-1-phenylsulfanylbutan-2-yl]amino]-3-nitrophenyl]sulfonylbenzamide (ABT-737), N-[4-(2-tert-butylphenyl)sulfonylphenyl]-2,3,4-trihydroxy-5-[(2-propan-2-ylphenyl)methyl]benzamide (TW-37), sabutoclax, obatoclax, maritoclax, gambogic acid, gossypol, motixafortide, cinobufagin, nodakenetin, mifepristone, A-1210477, A-1155463, AMG176, and AT101. Besides small molecules, other chemical entities, such as antisense RNAs, siRNAs, peptidyl inhibitors and antibody inhibitors of Bcl-2 can also be used. In some embodiments, the Bcl-2 inhibitor is venetoclax.

In some embodiments, the Bcl-2 family protein inhibitor is a Mcl-1 inhibitor. A variety of Mcl-1 inhibitors have been described, see for example, the compounds described in “Structure-Guided Design of a Series of MCL-1 Inhibitors with High Affinity and Selectivity” (Bruncko, et al., J. Med. Chem., 2015, 58 (5):2180-2194), “Small Molecule Mcl-1 Inhibitors for the Treatment of Cancer” (Belmar, et al., Pharmacol. Ther., 2015, 145:76-84), or “Small-Molecule Inhibitors of the Mcl-1 Oncoprotein” Chen, et al., Austin J. Anal. Pharm. Chem., 2014, 1(3). In some embodiments, the Mcl-1 inhibitor is selected from the group including, but not limited to, S63845, MIK665, 483-LM, AZD5991, AMG176, A1210477, MIM1, marinopyrrole A (maritoclax) BI97C10, BI112D1, gossypol, obatoclax, MG-132, sabutoclax, and TW-37. In some embodiments, the Bcl-2 family protein inhibitor is an inhibitor is a dual Bcl-2 inhibitor and Mcl-1 inhibitor.

In some embodiments, the Bcl-2 family protein inhibitor (e.g., the venetoclax) is used in combination with one of more additional active agents. For example, in some embodiments, the at least one additional active agent comprises or consists of venetoclax (or another Bcl-2 family protein inhibitor) and cytarabine (AraC). In some embodiments, the at least one additional active agent comprises or consists of venetoclax (or another Bcl-2 family protein inhibitor) and a hypomethylating agent. Hypomethylating agents are compounds that inhibit DNA methylation, e.g., by blocking the activity of DNA methyltransferase. In some embodiments, the hypomethylating agent is selected from azacytidine (AZA), cladribine, and decitabine. In some embodiments, the hypomethylating agent is AZA and/or decitabine. Thus, in some embodiments, the at least one additional active agent comprises or consists of venetoclax (or another Bcl-2 protein inhibitor) and AZA. In some embodiments, the at least one additional active agent comprises or consists of venetoclax (or another Bcl-2 protein inhibitor) and decitabine.

In some embodiments, the at least one additional active agent comprises or consists of AraC. In some embodiments, the at least one additional active agent comprises or consists of AraC and one or more of a Bcl-2 family protein inhibitor (e.g., a Bcl-2 protein inhibitor, such as venetoclax), daunorubicin (or another topoisomerase inhibitor), and a Hedgehog pathway inhibitor. Hedgehog pathway inhibitors include, but are not limited to, Smoothened (SMO) inhibitors, such as erismodegib, vismodegib, saridegib (IPI-926), BMS-833923/XL139, glasdegib (PF-04449919), and taladegib (LY2940680); GLI inhibitors, such as GANT-58 and Gant-61; and arsenic trioxide. In some embodiments, the Hedgehog pathway inhibitor is a SMO inhibitor. In some embodiments, the Hedgehog pathway inhibitor is glasdegib. Thus, in some embodiments, the at least one additional active agent comprises or consists of AraC and venetoclax, AraC and daunorubicin, or AraC and glasdegib. In some embodiments, the at least one additional agent comprises AraC and at least two of venetoclax, daunorubicin, and glasdegib.

In some embodiments, the at least one additional agent comprises or consists of a hypomethylating agent (HMA) (e.g., AZA, cladribine, and/or decitabine). In some embodiments, the HMA comprises or consists of decitabine and/or AZA.

In some embodiments, the at least one additional active agent comprises or consists of an antibody conjugate. For example, the antibody conjugate can comprise an antibody directed against a cell surface receptor found on myeloid cells (e.g., CD-33), where the antibody is covalently or non-covalently conjugated to a small molecule chemotherapeutic agent. In some embodiments, the antibody conjugate is gemtuzumab ozogamicin (sold under the brand name

Mylotarg), which is an antibody conjugate comprising an antibody to CD-33 covalently linked to a calicheamicin. Other antibody conjugates suitable for use according to the presently disclosed subject matter include, but are not limited to, CD123-targeting antibody drug conjugates (e.g., IMGN632 (see Kovtun et al., Blood Advances 2018, 2(8): 848-858)), CD13-targeting antibody drug conjugates, CLL-1-targeting antibody drug conjugates, and CD38 targeted antibody drug conjugates (see Williams et al., J. Clin Med 2019, 8: 1261). In some embodiments, the at least one additional active agent comprises or consists of an antibody conjugate (e.g., gemtuzumab ozogamicin) and a Hedgehog pathway inhibitor (e.g., glasdegib). In some embodiments, the at least one additional active agent comprises or consists of a Hedgehog pathway inhibitor, such as described hereinabove. In some embodiments, the at least one additional active agent comprises or consists of glasdegib.

Additional active agents that can also be included in the compositions of the presently disclosed subject matter include, but are not limited to FTL3 inhibitors, such as midostaurin, letaurtinib, pacritinib, and gliteritinib; and/or IDH1/2 inhibitors, such as ivosidenib and enasidenib. Thus, in some embodiments, the at least one additional active agent comprises or consists of one or more of midostaurin, letaurtinib, pacritinib, gliteritinib, ivosidenib, and enasidenib. In some embodiments, the FTL3 inhibitor and/or IDH1/2 inhibitor is used in combination with one or more of the other additional active agents, e.g., the Bcl-2 family protein inhibitor, AraC, or a HMA.

In some embodiments, the at least one additional active agent further comprises another therapeutic agent, such as a histone deacetylase (HDAC) inhibitor, and epigenetic regulator, and/or a histone demethylase inhibitor. HDAC inhibitors include hydroxamic acids, such as trichostatin A, virinostat (SAHA), belinostat (PXD101), LAQ824, and panobinostat (BLH589); benzamides, such as entinostat (MS-274), tacedinoline (CI994), and mocetinostat (MGCD0103); cyclic tetrapeptides, depsipetides, nicotinamide, dehydrocoumarin, naphthopyranone, and 2-hydroxynaphthaldehydes. Histone demethylase inhibitors include, but are not limited to TCP, ORY-1001, and GSK2879553. In some embodiments, the at least one additional active agent comprises (or further comprises in combination with a Bcl-2 family protein inhibitor, a HMA, or AraC) one or more of an IDH1/2 inhibitor (e.g., ivosidinib or enasidenib), a FTL3 inhibitor (e.g., midostaurin or gilteritinib), a HDAC inhibitor, an epigenetic regulator (e.g., cladribine), and a histone demethylase inhibitor.

In some embodiments, the presently disclosed subject matter provides a method for treating AML. In some embodiments, the method comprises administering to a subject in need thereof a composition comprising (a) an AC inhibitor; and (b) at least one additional active agent, wherein the composition is administered via a route and in an amount effective for treating the AML. In some embodiments, the at least one additional active agent includes at least one of (e.g., at least one, at least two, at least three, at least four, or at least five) of the group including a CNL, an inhibitor of a Bcl-2 family protein, a hypomethylating agent, cytarabine (AraC), daunorubicin, a Hedgehog pathway inhibitor, a FTL3 inhibitor, an IDH1/2 inhibitor, and an antibody conjugate, wherein the composition is administered via a route and in an amount effective for treating the AML. In some embodiments, the AML is a newly diagnosed AML. In some embodiments, the AML is RR-AML.

Suitable AC inhibitors, CNLs, Bcl-2 family protein inhibitors (e.g., Bcl-2 and/or Mcl-1 inhibitors), hypomethylating agents, Hedgehog pathway inhibitors, FTL3 inhibitors, IDH1/2 inhibitors, and antibody conjugates for use in the method are described hereinabove. In some embodiments, the AC inhibitor comprises or consists of SACLAC or a pharmaceutically acceptable salt thereof.

In some embodiments, the at least one additional active agent comprises or consists of a CNL. In some embodiments, the CNL is a C6-CNL.

In some embodiments, the at least one additional active agent comprises or consists of a Bcl-2 family protein inhibitor. In some embodiments, the Bcl-2 family protein inhibitor comprises or consists of a Bcl-2 inhibitor. In some embodiments, the Bcl-2 inhibitor comprises or consists of venetoclax. In some embodiments, the Bcl-2 family protein inhibitor is a Mcl-1 inhibitor. In some embodiments, the at least one additional active agent comprises or consists of venetoclax (or another Bcl-2 family inhibitor) and AraC and/or a hypomethylating agent (e.g., AZA or decitabine). In some embodiments, the hypomethylating agent comprises or consists of AZA. Thus, in some embodiments, the presently disclosed subject matter provides a method of treating AML via administration of a composition comprising or consisting of an AC inhibitor (e.g., SACLAC), a Bcl-2 family protein inhibitor (e.g., venetocax) and AraC. In some embodiments, the presently disclosed subject matter provides a method of treating AML via administration of a composition comprising or consisting of an AC inhibitor (e.g., SACLAC), a Bcl-2 family inhibitor (e.g., venetocax) and one or both of AZA and decitabine.

In some embodiments, the at least one additional active agent comprises or consists of AraC. In some embodiments, the at least one additional active agent comprises AraC in combination with one or more other additional active agent. In some embodiments, the AraC is used in combination with at least one of venetoclax (or another Bcl-2 family inhibitor), daunorubicin, and a Hedgehog pathway inhibitor (e.g., glasdegib). Thus, in some embodiments, the at least one additional active agent comprises or consists of AraC and glasdegib.

In some embodiments, the at least one additional active agent comprises or consists of a hypomethylating agent. In some embodiments, the hypomethylating agent comprises or consists of decitabine and/or AZA.

In some embodiments, the at least one additional active agent comprises or consists of an antibody conjugate and/or a Hedgehog pathway inhibitor. In some embodiments, the antibody conjugate is gemtuzumab ozogamicin. In some embodiments, the Hedgehog pathway inhibitor is glasdegib.

In some embodiments, the at least one additional active agent comprises or consists of a FTL3 inhibitor and/or an IDH1/2 inhibitor. In some embodiments, the at least one additional active agent comprises or consists of one or more of ivosidenib, enasidenib, midostaurin, and gilteritinib. In some embodiments, the FTL3 inhibitor and/or IDH1/2 inhibitor is used in combination with at least one of the other additional active agents, e.g., a Bcl-2 family protien inhibitor, AraC, or an HMA. In some embodiments, the at least one additional active agent can further comprise a MAC inhibitor, an epigenetic regulator, and a histone demethylase inhibitor.

In some embodiments, one or more components of the composition are encapsulated in a polymer. For example, one or more component can be encapsulated in a nanoparticle comprising a biodegradable and/or biocompatible polymer, such as poly(ethylene glycol) (PEG; which can also be referred to as poly(ethylene oxide) (PEO)), poly(lactic-co-glycolic acid) (PLGA), poly(d,l-lactic acid) (PDLLA), or a copolymer thereof. In some embodiments, one or more components are encapsulated in a PEG-PDLLA diblock copolymer or a PEG-PLGA copolymer. In some embodiments, the AC inhibitor is encapsulated in a polymer nanoparticle (e.g., a PEG-PDLLA diblock copolymer nanoparticle or PEG-PLGA copolymer).

In some embodiments, the AML, treated by a method disclosed herein is a drug-resistant or drug-sensitive AML. In some embodiments, the AML is an AML that is resistant to one or more AML treatment agents. In some embodiments, the AML is a venetoclax-resistant AML.

As used herein, a “subject” of treatment is typically an animal. Such animals include mammals, such as animals of economic importance, e.g., livestock like horses, donkeys, mules, cattle, goats, sheep, llama, alpaca, etc.; animals of social importance, such as those kept as pets, such as cats, dogs, rabbits, hamsters, guinea pigs, mice, rats, etc., and those that are endangered or kept in zoos, such as zebra, elephants, lions, tigers, bears, etc. As used herein, a “subject in need thereof” is a patient, animal, mammal, or human, who will benefit from the methods and compositions of the presently disclosed subject matter. Thus, in some embodiments the subject is a mammal, optionally a human.

In some embodiments, the components of the compositions used in the instant methods, e.g., the AC inhibitor and the one or more additional active agent, can be provided in a single formulation or composition. However, they can also be provided in multiple individual compositions or formulations or in compositions or formulations comprising subsets of the components. In some embodiments, the components can be administered at about the same time (e.g., in the same composition or in multiple compositions). In some embodiments, the components can be administered sequentially, e.g., over the course of one or more hours, days, weeks, or months.

In some embodiments, the presently disclosed subj ect matter provides a composition for use in treating a disease, disorder, or condition associated with an AC biological activity. In some embodiments, the composition comprises (i) an AC inhibitor (e.g., SACLAC or a pharmaceutically acceptable salt thereof or another AC inhibitor as described hereinabove), and (ii) at least one additional active agent. In some embodiments, at least one additional active agent comprises at least one of (e.g., one, two, three, four, five, six, or more of) a CNL, an inhibitor of a Bcl-2 family protein, a hypomethylating agent, AraC, daunorubicin, a Hedgehog pathway inhibitor, a FTL3 inhibitor, an IDH1/2 inhibitor, and an antibody conjugate. The composition can comprise the components (i.e., the AC inhibitor and the at least one additional active agent) in an amount effective for reducing the AC biological activity in a subject.

In some embodiments, the presently disclosed subj ect matter provides a composition for use in treating AML. In some embodiments, the composition comprises (i) an AC inhibitor (e.g., SACLAC or a pharmaceutically acceptable salt thereof or another AC inhibitor as described hereinabove), and (ii) at least one additional active agent. In some embodiments, at least one additional active agent comprises at least one of (e.g., one, two, three, four, five, six, or more of) a CNL, an inhibitor of a Bcl-2 family protein, a hypomethylating agent, AraC, daunorubicin, a Hedgehog pathway inhibitor, a FTL3 inhibitor, an IDH1/2 inhibitor, and an antibody conjugate. The composition can comprise the components (i.e., the AC inhibitor and the at least one additional active agent) in an amount effective for treating AML in a subj ect (e.g., a mammalian subject, such as a human). The composition can be used for treating newly diagnosed AML or RR-AML. In some embodiments, the AML is a drug-resistant AML, such as venetoclax-resistant AML.

In some embodiments, the at least one additional active agent comprises or consists of venetoclax, AraC, and/or AZA. Thus, in some embodiments, the composition can comprise or consist of SACLAC (or another AC inhibitor) and venetoclax. In some embodiments, the composition can comprise or consist of SACLAC (or another AC inhibitor), venetoclax and AraC. In some embodiments, the composition can comprise or consist of SACLAC (or another AC inhibitor), venetoclax and a hypomethylating agent (e.g., AZA). In some embodiments, any of these compositions can further include a CNL (e.g., a C-6 CNL). In some embodiments, the at least one additional active agent comrpises daunorubicin, glasdegib, and/or gemtuzumab ozogamicin. In some embodiments the composition comprises of consists of SACLAC (or another AC inhibitor), AraC and glasdegib.

In some embodiments, the composition can comprise or consists of one or more of ivosidenib, enasidenib, midostaurin, gilteritinib. In some embodiments, the composition can further comprise one or more of a HDAC inhibitor, an epigenetic regulator, and a histone demethylase inhibitor.

In some embodiments, the presently disclosed subject matter provides a method of enhancing the delivery of an AC inhibitor (e.g., SACLAC). In some embodiments, the AC inhibitor is administered in a CNL liposome (e.g., a C6-liposome). In some embodiments, the AC inhibitor is administered encapsulated in a polymer nanoparticle. Suitable polymers of use as the encapsulating nanoparticle comprises biodegradable and/or biocompatible polymers. In some embodiments, the polymer is both biodegradable and biocompatible. In some embodiments, the polymer is a combination of PEG (or PEO) and PDLLA or PLGA. In some embodiments, the polymer is a PEG-PDLLA copolymer or a PEG-PLGA copolymer. In some embodiments, the encapsulated AC inhibitor is SACLAC. In some embodiments, providing the AC inhibitor in a polymeric nanoparticle improves the potency (e.g., lowers the 50% effective concentration) of the AC inhibitor compared to when the AC inhibitor is administered in a non-encapsulated form. In some embodiments, administering the AC inhibitor in an encapsulated from improves potency by about 1.4 times or more. In some embodiments, administering the AC inhibitor in an encapsulated form improves the potency of the AC inhibitor by about 3 times or more.

In some embodiments, one or more other agents for treating an AC activity-related disease or disorder (e.g., AML) are also encapsulated in the polymer nanoparticle. In some embodiments, the one or more other agents include, but are not limited to, an inhibitor of a Bcl-2 family protein, a hypomethylating agent, AraC, daunorubicin, a Hedgehog pathway inhibitor, a FTL3 inhibitor, an IDH1/2 inhibitor a HDAC inhibitor, an epigenetic regulator, and a histone demethylase inhibitor.

In some embodiments, the presently disclosed subj ect matter provides a composition for use in treating a disease, disorder or condition associated with an AC activity (e.g., AML), wherein the composition comprises a AC inhibitor (optionally a ceramide analog AC inhibitor such as SACLAC), wherein the AC inhibitor is encapsulated in a CNL or a biodegradable polymer. In some embodiments, the biodegradable polymer is a copolymer of PEG and PGLA or PDLLA. In some embodiments, the AC inhibitor is SACLAC.

III.A. Liposomes

In some embodiments, the presently disclosed subject matter provides for the use of compositions comprising liposomes (e.g., CNLs or other liposomes). Liposomes can be prepared by any of a variety of techniques that are known in the art. See e.g., Betageri et al. (1993) Liposome Drug Delivery Systems, Technomic Publishing, Lancaster, Pa., United States of America; Gregoriadis, ed. (1993) Liposome Technology, CRC Press, Boca Raton, Fla., United States of America; Janoff, ed. (1999) Liposomes: Rational Design, M. Dekker, New York, N.Y., United States of America; Lasic & Martin (1995) Stealth Liposomes, CRC Press, Boca Raton, Florida, United States of America; and U.S. Pat. Nos. 4,235,871; 4,551,482; 6,197,333; and 6,132,766, each of which is incorporated herein by reference in its entirety. Temperature-sensitive liposomes can also be used, for example THERMOSOMES™ as disclosed in U.S. Pat. No. 6,200,598, which is incorporated herein by reference in its entirety. Entrapment of an active agent within liposomes of the presently disclosed subject matter can also be carried out using any conventional method in the art. In preparing liposome compositions, stabilizers such as antioxidants and other additives can be used.

Other lipid carriers can also be used in accordance with the presently disclosed subject matter, such as lipid microparticles, micelles, lipid suspensions, and lipid emulsions. See, e.g., Labat-Moleur et al. (1996) An electron microscopy study into the mechanism of gene transfer with lipopolyamines. Gene Therapy 3:1010-1017; U.S. Pat. Nos. 5,011,634; 6,056,938; 6,217,886; 5,948,767; and 6,210,707, each of which is incorporated herein by reference in its entirety.

In some embodiments, a liposome is a ceramide nanoliposome (CNL). CNLs are described in U.S. Pat. No. 8,747,891 and U.S. Patent Application Publication No. 2019/0031756, each of which is incorporated herein in its entirety. In some embodiments, the CNL encompasses the one or more short chain ceramides (including but not limited to C6 ceramides) and one or more chemotherapeutically active agents.

Delivery time frames can be provided according to a desired treatment approach. By way of example and not limitation, the first delivery vehicle can deliver substantially all of the provided active agent within 24 hours after administration wherein the second delivery vehicle can deliver a certain much smaller amount within the first 24 hours, first 3 days, first week, and substantially all within the first 2, 3, 4, 5, 6, or 7 weeks, as desired. Thus, the duration of the delivery can be altered with the chemistry of the delivery vehicle.

The delivery vehicles can comprise nano-, submicron-, and/or micron-sized particles. In some embodiments, the delivery vehicles are about 50 nm to about 1 μm in their largest dimensions. Thus, in some embodiments the delivery vehicle can comprise a nanoparticle, a microparticle, or any combination thereof. As used herein, the terms “nano”, “nanoscopic”, “nanometer-sized”, “nanostructured”, “nanoscale”, and grammatical derivatives thereof are used synonymously and interchangeably and mean nanoparticles and nanoparticle composites less than or equal to about 1,000 nanometers (nm) in diameter. Similarly, the terms “micro”, “microscopic”, “micrometer-sized”, “microstructured”, “microscale”, and grammatical derivatives thereof are used synonymously and interchangeably and mean microparticles and microparticle composites that are larger than 1,000 nanometers (nm) but less than about 5, 10, 25, 50, 100, 250, 500, or 1000 micrometers in diameter.

The term “delivery vehicle” as used herein thus denotes a carrier structure which is biocompatible with and sufficiently resistant to chemical and/or physical destruction by the environment of use such that a sufficient amount of the delivery vehicles remain substantially intact after deployment at a site of interest. If the active agent is to enter a cell, tissue, or organ in a form whereby it is adsorbed to the delivery vehicle, the delivery vehicle must also remain sufficiently intact to enter the cell, tissue, or organ. Biodegradation of the delivery vehicle is permissible upon deployment at a site of interest.

As used herein, the term “biodegradable” means any structure, including but not limited to a nanoparticle, which decomposes or otherwise disintegrates after prolonged exposure to physiological conditions. To be biodegradable, the structure should be substantially disintegrated within a few weeks after introduction into the body.

Biodegradable biocompatible polymers can be used in drug delivery systems (Soppimath et al., (2001) Biodegradable polymeric nanoparticles as drug delivery devices. J Controlled Release 70:1-20; Song et al. (1997) Formulation and characterization of biodegradable nanoparticles for intravascular local drug delivery. J Controlled Release 43:197-212; U.S. Patent Application Publication Nos. 2011/0104069, 2013/0330279, 2018/0078657, 2019/0091280, and 2020/0038452, and U.S. Pat. Nos. 7,332,586; 7,901,711; 8,137,697; 8,449,915; and 8,663,599, each of which is incorporated herein by reference in its entirety). The biodegradability and biocompatibility of poly(lactic acid) (PLA), poly(lactide-co-glycolide) (PLGA), and polyanhydrides (PAH) have been demonstrated. Some of the advantages of these materials include administration in high concentrations of the drug locally with low systemic levels, which reduces systemic complications and allergic reactions (Calhoun et al. (1997) Treatment of osteomyelitis with a biodegradable antibiotic implant. Clin Orthopaed Related Res 341:206-214). Additionally, no follow-up surgical removal is required once the drug supply is depleted (Mandal et al. (2002) Poly(D,L-lactide-co-glycolide) encapsulated poly(vinyl alcohol) hydrogel as a drug delivery system. Pharmaceut Res 19:1713-1719). Biodegradation occurs by simple hydrolysis of the ester backbone in aqueous environments such as body fluids. The degradation products are then metabolized to carbon dioxide and water (de Faria et al. (2005) Preparation and Characterization of Poly(D,L-Lactide) (PLA) and Poly(D,L-Lactide)-Poly(Ethylene Glycol) (PLA-PEG) Nanocapsules Containing Antitumoral Agent Methotrexate. Macromol Symp 229:228-233). Several techniques have been developed to prepare nanoparticles loaded with a broad variety of drugs using PLGA and to some extent with PAH (Lamprecht et al. (1999) Biodegradable monodispersed nanoparticles prepared by pressure homogenization-emulsification. Intl J Pharmaceut 184:97-105; Astete et al. (2006) Synthesis and characterization of PLGA nanoparticles. J Biomater Sci Polymer Edn 17:247-289; Hans et al. (2002) Synthesis and characterization of mPEG-PLA prodrug micelles. Solid State Mater Sci 6:319-327., 2002; Kumar et al. (2004) Preparation and characterization of cationic PLGA nanospheres as DNA carriers. Biomaterials 25:1771-1777; Laurencin et al. (2001) Poly(lactide-co-glycolide)/hydroxyapatite delivery of BMP-2-producing cells: a regional gene therapy approach to bone regeneration. Biomaterials 22:1271-1277; Gonsalves et al. (1998) Synthesis and surface characterization of functionalized polylactide copolymer microparticles. Biomaterials 19:1501-1505; et al. (2001) Preparation of PLGA nanoparticles containing estrogen by emulsification—diffusion method. Colloids Surf A 182:123-130).

In some embodiments, the composition can comprise a pharmaceutically acceptable carrier, diluent, or excipient. As used herein, the term “pharmaceutically acceptable” and grammatical variations thereof, as it refers to compositions, carriers, diluents and reagents, means that the materials are capable of administration to or upon a vertebrate subject without the production of undesirable physiological effects such as nausea, dizziness, gastric upset, fever and the like. In some embodiments, the “pharmaceutically acceptable” refers to pharmaceutically acceptable for use in human beings. In some embodiments, the diluent can comprise β-HPCD (2-hydroxypropyl β-cyclodextrin).

Compositions in accordance with the presently disclosed subject matter generally comprise an amount of the desired delivery vehicle (which can be determined on a case-by-case basis), admixed with an acceptable pharmaceutical diluent or excipient, such as a sterile aqueous solution, to give an appropriate final desired concentration in accordance with the dosage information set forth herein, and/or as would be apparent to one of ordinary skill in the art upon a review of the instant disclosure, with respect to the antibiotic. Such formulations will typically include buffers such as phosphate buffered saline (PBS), or additional additives such as pharmaceutical excipients, stabilizing agents such as BSA or HSA, or salts such as sodium chloride. Such components can be chosen with the preparation of composition for local, and particularly topical, administration in mind.

III.B. Formulations

The compositions of the presently disclosed subject matter can be administered in any formulation or route that would be expected to deliver the compositions to the subjects and/or target sites present therein.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject. For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

III.C. Routes of Administration

By way of example and not limitation, suitable methods for administering a composition in accordance with the methods of the presently disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see e.g., U.S. Pat. No. 6,180,082, which is incorporated herein by reference in its entirety). In some embodiments, a composition comprising a nanoparticle and/or an exosome is administered orally.

Thus, exemplary routes of administration include parenteral, enteral, intravenous, intraarterial, intratumoral, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated.

III.D. Dose

An effective dose of a composition of the presently disclosed subject matter is administered to a subject in need thereof. An “effective amount” or a “therapeutic amount” is an amount of a composition sufficient to produce a measurable response. Exemplary responses include biologically or clinically relevant responses in subj ects such as but not limited to an improvement in a symptom. Actual dosage levels of the compositions of the presently disclosed subject matter can be varied so as to administer an amount of the composition that is effective to achieve the desired response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subj ect matter at levels lower than required to achieve the desired therapeutic effect and to gradually increase the dosage until the desired effect is achieved. The potency of a composition can vary, and therefore an “effective amount” can vary. However, using the methods described herein, one skilled in the art can readily assess the potency and efficacy of a composition of the presently disclosed subject matter and adjust the regimen accordingly.

As such, after review of the instant disclosure, one of ordinary skill in the art can tailor the dosages to an individual subject, taking into account the particular formulation, method of administration to be used with the composition, and particular disease, disorder, and/or condition treated or biologically relevant outcome desired. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art.

Typically, a suitable dose can be in the range of from about 0.5 to about 100 mg/kg, e.g., from about 10 to about 75 mg/kg of body weight per day, such as 3 to about 50 mg per kilogram body weight of the recipient per day, in the range of 6 to 90 mg/kg/day, or in the range of 15 to 60 mg/kg/day.

The compound can be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four, or more sub-doses per day. The sub-dose itself may be further divided, e.g., into a number of discrete loosely spaced administrations.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Materials and Methods

### AML Cell Lines and Patient Samples

Cell lines: Kasumi-1, Kasumi-3, Kasumi-6, ME-1, SET2 and SKM-1 cells were cultured in RPMI-1640 (#10-040; Corning Inc., Corning, New York, United States of America) with 20% FBS (#97068-085, VWR International, Radnor, Pa., United States of America)). OCI-AML3 cells were cultured in RPMI-1640 with 15% FBS. OCI-AML4 cells were cultured in a-MEM (#12571063, ThermoFisher Scientific, Waltham, Massachusetts, United States of America) with 20% FBS and supplemented with 100 ng/ml hGM-CSF (Milltenyi Biotec #130-095-372, Milltenyi Biotec, Bergisch Gladbach, Germany). All KG1 derivative cells and OCI-M2 cells were cultured in IMDM (#12440, ThermoFisher Scientific, Waltham, Mass., United States of America) with 20% FBS. All other cell lines were cultured in RPMI-1640 media supplemented with 10% FBS.

HL-60/VCR (1) cells were maintained in the presence of 1 mg/ml vincristine sulfate (#11764, Cayman Chemical Company, Ann Arbor, Michigan, United States of America). HL-60/ABTR (2) cells were maintained in the presence of 5μM ABT-737 (#11501, Cayman Chemical Company, Ann Arbor, Michigan, United States of America). KG1/ABTR and KG1a/ABTR (2) cells were maintained in the presence of 1μM ABT-737. Kasumi-6 and SNKO1 cells were supplemented with 10 ng/ml hGM-CSF. TF-1 cells were supplemented with 2 ng/ml hGM-CSF.

OCI-M2, SKNO1 and SKM-1 cell lines were obtained from the Liebniz Institute-DSMZ (German Collection of Microorganisms and Cell Cultures, GmbH; Braunshweig, Germany). All other cell lines were obtained from the American Type Culture Collection (ATCC, Manassas, Va., United States of America) or gifted. All cells were grown at 37° C. and 5% CO₂ in a humidified incubator. Cell lines were authenticated by short tandem repeat DNA profiling (Genetica DNA Laboratories; Lab Corp of America, Burlington, North Carolina, United States of America) and tested for mycoplasma contamination routinely using a detection kit sold under the tradename MYCOALERTTM PLUS (#LT07-710, Lonza Group AG, Basel, Switzerland). Experiments were performed within 6 weeks of thawing.

HL-60/VCR, OCI-AML2 and THP-1 cell lines were chosen for characterization studies based on prevalence in literature and ease of maintenance. KGla cells were chosen for siRNA studies based on transfection efficiency with published electroporation parameters. Table 1 below provides RRID numbers.

AML patient samples: Samples were prepared from peripheral blood collected from newly diagnosed and untreated AML, patients. Mononuclear cells were isolated by Ficoll-Paque (GE Healthcare Life Sciences, Piscataway, N.J., United States of America) density gradient centrifugation. Cells were cultured in a serum-free medium sold under the tradename STEMSPAN™ SFEM (referred to as SFEM) purchased from Stem Cell Technologies (Vancouver, Canada) and supplemented with recombinant human stem cell factor (SCF, 100 ng/ml), interleukin-3 (IL-3, 20 ng/ml), FMS-like tyrosine kinase ligand (FLT3L, 100 ng/ml), granulocyte colony-stimulating factor (G-CSF, 20 ng/ml) and granulocyte-macrophage colony-stimulating factor (GM-CSF, 20 ng/ml) (Shenandoah Biotechnology Inc., Warwick, Pennsylvania, United States of America). All cultures were incubated at 37° C. with 5% CO₂. Informed consent was obtained from all patients.

Healthy donor samples: PBMCs from healthy donors were enriched using the Ficoll-Paque gradient separation method. CD34+PBMCs mobilized with G-CSF were obtained from the University of Virginia Health System Blood Bank (Charlottesville, Va., United States of America). CD34+ cells were isolated from PBMCs with a Human CD34 MicroBead Kit (#130-046, Miltenyi Biotec, Bergisch Gladbach, Germany) using the autoMACS Pro Separator (#130-092-545, Miltenyi Biotec, Bergisch Gladbach, Germany).

TABLE 1
RRID numbers.

| Cell Line | RRID |
|---|---|
| EOL-1 | CVCL_0258 |
| HEL | CVCL_2481 |
| HL-60 | CVCL_0002 |
| HL-60/ABTR | N/A |
| HL-60/VCR | CVCL_0305 |
| Kasumi-1 | CVCL_0589 |
| Kasumi-3 | CVCL_0612 |
| Kasumi-6 | CVCL_0614 |
| KG1 | CVCL_0374 |
| KG1/ABTR | N/A |
| KG1a | CVCL_1824 |
| KG1a/ABTR | N/A |
| ME-1 | CVCL_2110 |
| ML2 | CVCL_1418 |
| MM-6 | CVCL_1426 |
| MOLM-13 | CVCL_2119 |
| MOLM-14 | CVCL_7916 |
| MV4-11 | CVCL_0064 |
| NB4 | CVCL_0005 |
| NOMO1 | CVCL_1609 |
| OCI-AML2 | CVCL_1619 |
| OCI-AML3 | CVCL_1844 |
| OCI-AML4 | CVCL_5224 |
| OCI-M2 | CVCL_2150 |
| SET2 | CVCL_2187 |
| SKM1 | CVCL_0098 |
| SNKO1 | CVCL_2196 |
| TF-1 | CVCL_0559 |
| THP-1 | CVCL_0006 |
| U937 | CVCL_0007 |

### Compounds

SACLAC N-[(2S,3R)-1,3-dihydroxyoctadecan-2-yl]2-chloroacetamide was synthesized as previously described (33). The structure was validated using mass spectrometry. 2-hydroxypropyl β-cyclodextrin was obtained from Acros Organics (#297561000, Acros Organics BVBA, Fair Lawn, N.J., United States of America). ABT-737 and ceramides mixture were obtained from Cayman Chemicals (#11501 and #22853, respectively, Cayman

Chemicals Company, Ann Arbor, Mich., United States of America). The ceramide mixture contained trace amount of C16 ceramide and varying amounts of longer chain and 2-hydroxy ceramides with C24 species being most abundant. C16 was purchased from Avanti Polar Lipids (#860516, Avanti Polar Lipids, Alabaster, Ala., United States of America). All ceramides were dissolved in methanol with 2% dodecane. LCL204 was synthesized according to previously published methods (3). RBM14C12 substrate for AC activity assays is commercially available (e.g., from Avanti Polar Lipids, Alabaster, Alabama, United States of America).

### Acid Ceramidase Activity Assay

Cells were seeded at 2×10⁴ cells per well in 50 μl Acid ceramidase activity was measured at 24 hours using the RBM14C12 fluorogenic substrate as previously described (22,34).

### Sphingolipid Analysis

Lipid extraction and analysis was done using liquid chromatography-electrospray ionization-tandem mass spectrometry (LC-ESI-MS/MS) as previously described (22). Cells were plated at 5 million cells per 6 ml and treated with SACLAC or DMSO for 24 hours. Lipids were extracted from cell pellets using an azeotrophic mix of isopropanol: water: ethyl acetate (3:1:6; v:v:v). Internal standards (50 pmol of d17 long-chain bases and C12 acylated sphingolipids) were added to samples at the onset of the extraction procedure. Extracts were separated on a Waters I-class Acquity UPLC chromatography system (Waters Corporation, Milford, Mass., United States of America). Mobile phases were (A) 60:40 water: acetonitrile and (B) 90:10 isopropanol:methanol with both mobile phases containing 5 mM ammonium formate and 0.1% formic acid. A Waters C18 CSH 2.1 mm ID×10 cm column maintained at 65° C. was used for the separation of the sphingoid bases, 1-phosphates, and acylated sphingolipids. The eluate was analyzed with an inline Waters TQ-S mass spectrometer (Waters Corporation, Milford, Mass., United States of America) using multiple reaction monitoring. All data reported are based on monoisotopic mass and are represented as pmol/mg protein.

### Cell Viability Assay

For cell viability, cell lines were plated at 2.5×10⁴ cells per well in a 96-well plate and treated with SACLAC or DMSO vehicle (0.4% of total volume) for the indicated time points and doses. At the experiment end point, 3-(4,5-Dimethylthiazol-2-yl)-5-(3-carboxymethoxyphenyl)-2-(4-sulfophenyl)-2H-tetrazolium (MTS) Cell Proliferation Colorimetric Assay Kit (# K300-5000, BioVision, Inc., Milpitas, Calif., United States of America) reagent was added and incubated for 2 hours. Conversion of MTS to formazan product was measured by absorbance at 490 nm using a BioTek Cytation 3 plate reader (BioTeck, Winooski, Vt., United States of America). Absorbance was normalized to DMSO control, which was defined as 100% viability. Due to limited cell numbers, patient samples and normal samples were plated at 5×10³ cells per well in a 384-well plate with SACLAC or DMSO control for the indicated time points and doses. At the experiment end point, a luminescent reagent sold under the tradename CELLTITER-GLO™ (#G7572, Promega Corporation, Madison, Wis., United States of America) was added. After 15 minutes, luminescence was read on a BioTek Cytation 3 plate reader (BioTek, Winooski, Vt., United States of America). Absorbance was normalized to DMSO control, which was defined as 100% viability.

### Colony Forming Assay

Cryopreserved human AML patient samples were thawed and washed twice with RPMI 1640 supplemented with 2% heat inactivated fetal bovine serum (FBS). After washes, cells were cultured in triplicate in 12-well plates at a density of 0.1 to 2×10⁵ cells per well in Human Methylcellulose Complete Media (#HSC003R&D Systems, Minneapolis, Minnesota, United States of America). Plating densities were selected for each case to yield colony outgrowth of 20-100 colonies per well. The desired cell number and dose of SACLAC or DMSO was added to the culture media and dispensed to multi-well plates. Colonies were propagated for 10-14 days and blast colonies (>20 cells) were counted in a blinded manner under the light microscope.

### Flow Cytometry

Apoptosis in cell lines was assessed after treating 2.5×10⁵ cells per ml with drug or vehicle in a 48-well plate for the indicated time points and doses. Primary human AML cells were pre-incubated with SFEM for 48 hours before plating 2×10⁵ cells in 24-well plates for the indicated times point and doses. Samples were stained using the Muse Annexin V & Dead Cell Kit (Millipore #MCH100105). Change in mitochondrial membrane potential in cell lines was measured with the Muse® MitoPotential Kit (#MCH100110, MilliporeSigma, Burlington, Mass., United States of America). Caspase activation was measured using the Muse® Caspase 3/7 Assay Kit (#MCH100108, MilliporeSigma, Burlington, Mass., United States of America). For rescue experiments, cells were plated at 2.5×10⁴ well in a 96-well plate 24 hours after electroporation. Cells were treated with DMSO or SACLAC for 48 hours and apoptosis was detected as stated above. All kits were used according to manufacturer's protocol. Cells were then analyzed using the Muse® Cell Analyzer (MilliporeSigma, Burlington, Massachusetts, United States of America) (35,36). Experiments included positive and negative controls for proper analysis.

### Western Blotting

Cells were plated at 2.5×10⁵ cells per ml in 6-well plates and treated with drug, vehicle or siRNA at the indicated doses and time points. Cells were harvested, washed with PBS and lysed prior to preparation of protein extracts for western blotting.

More particularly, cells were lysed with RIPA buffer (#R0278, Sigma, St. Louis, Mo., United States of America) containing phosphatase inhibitor cocktails 2 and 3 (Sigma #P5726, #P0044) and protease inhibitor cocktail (#P8340, Sigma, St. Louis, Mo., United States of America). Protein was quantified using a bicinchoninic acid (BCA) protein assay kit (#23225, Pierce, Waltham, Mass., United States of America). Samples were resolved on a Bolt 4-12% SDS-PAGE gel (#NW00082, ThermoFisher Scientific, Waltham, Mass., United States of America) and transferred to PVDF membrane (#170-4274, Bio-Rad Laboratories, Hercules, Calif., United States of America). Antibodies were obtained from Cell Signaling Technology (Danvers, Mass., United States of America) unless indicated otherwise. Primary antibodies used were: Mcl-1 (#5453, RRID #AB 10694494), SF3B1 (#14434, RRID #AB 2798479), β-actin (#3700, RRID #AB_2242334) and AC (BD Biosciences #612302 (San Jose, California, United States of America), RRID #AB_399617). Secondary antibodies used were HRP-linked goat anti-mouse (#7076, RRID #AB_330924) or goat anti-rabbit IgG (#7074, RRID # AB_2099233). Clarity Max Western ECL Substrate (#1705062, Bio-Rad Laboratories, Hercules, Calif., United States of America) was added to visualize relative protein expression by chemiluminescence using the Bio-Rad ChemiDoc MP imaging system (Bio-Rad Laboratories, Hercules, Calif., United States of America). Quantification was done using Bio-Rad ImageLab 6.0.1 software (Bio-Rad Laboratories, Hercules, Calif., United States of America). For quantification, bands were normalized to β-actin as a loading control.

Transfection with siRNA by Electroporation

KG1a cells were electroporated with non-targeting scrambled siRNA (#D-001810-10-20, Dharmacon, Lafayette, Colo., United States of America), siRNA targeting ASAH1 (#L-005228-03-0010, Dharmacon, Lafayette, Colorado, United States of America) or MCL-1S (#CTM-481502, Dharmacon, Lafayette, Colo., United States of America) using the Neon Transfection System (Invitrogen, Carlsbad, Calif., United States of America) according to the manufacturer's protocol with the following parameters: 3×10⁷ cells per ml, 1700 pulse voltage, and 20 ms pulse width for a single pulse. AC knockdown cells were harvested 48 hours after transfection for analysis by western blot. Mcl-1S knockdown cells were re-plated to 2.5×10⁵ cells per ml and treated with SACLAC 24 hours after electroporation. Control- and SACLAC-treated cells were harvested 48 hours after SACLAC treatment, corresponding to 72 hours after electroporation.

Overexpression with cDNA Via Electroporation

KG1a cells were electroporated with empty vector, an M98 vector containing Mcl-1 cDNA (NM_021960.4; Genecopoeia, Rockville, Md., United States of America) or a pCMV vector containing a synthetic sequence for WT SF3B1 (37) using the same protocol listed above.

### Quantitative Reverse Transcription PCR

Cells were seeded at 2.5×10⁵ cells per ml in 6-well plates and treated with DMSO or SACLAC for six hours. Cells were harvested and resuspended in TRIzol reagent (#15596026, Invitrogen, Carlsbad, Calif., United States of America). RNA was isolated using Direct-zol RNA Miniprep Plus (#R2072, Zymo Research, Irvine, Calif., United States of America) according to the manufacturer's protocol. RNA was quantified using the Take3 microplate and the pre-programmed RNA quantification protocol on the Gen5 software for the Cytation 3 plate reader (BioTek, Winooski, Vt., United States of America). DNase treatment and reverse transcription were done using the iScript gDNA Clear cDNA Synthesis Kit (#1725035, Bio-Rad Laboratories, Hercules, Calif., United States of America) in accordance with the manufacturer's protocol. The qPCR reaction contained iTaq Universal SYBR Green (#1725122, Bio-Rad Laboratories, Hercules, Calif., United States of America), 140 ng cDNA and primers specific for HPRT (#100-25636, Bio-Rad Laboratories, Hercules, Calif., United States of America), B2M (#100-25636, Bio-Rad Laboratories, Hercules, Calif., United States of America) and MCL-1S (Eurofins (Luxembourg); forward, 5′-GAGGAGGACGAGTTGTACCG-3′ (SEQ ID NO: 1) and reverse, 5′-ACTCCACAAACCCATCCTTG-3′ (SEQ ID NO: 2)) (38). This primer pair specifically amplifies Mcl-1 transcript variant 2 (MCL-1S) according to BLAST analysis. CT values were normalized using B2M and HPRT housekeeping genes and transformed to 2^{-ΔCT} and the DMSO control was set to 1.

### In Vivo AML Xenograft Model

MV4-11 AML cells expressing luciferase (luc2) and YFP (39) or U937 AML cells expressing luc2 and tdTomato (#72486, Addgene, Watertown, Mass., United States of America) were used to investigate the efficacy of SACLAC in NOD-scid IL2Rgammana (NSG) mice (Jackson Laboratories, Bar Harbor, Me., United States of America). Maximum tolerated dose and pharmacokinetic studies were also performed to determine safety and delivery of compound.

More particularly, maximum tolerated dose of SACLAC was determined in NOD-scid IL2Rgamma^{null} (NSG) mice (n=5; Jackson Laboratories, Bar Harbor, Me., United States of America). Mice were injected with SACLAC reconstituted in β-HPCD (IV) daily for 5 days at a dose of 5 mg/kg body weight, respectively. For pharmacokinetics studies, Swiss Webster mice (n=3; Charles River Laboratories, Wilmington, Mass., United States of America) were injected with SACLAC dissolved in DMSO (40 mg/kg body weight, intraperitoneally) or dissolved in β-HPCD (5 mg/kg body weight, via tail-vein injection). Mice were euthanized and blood was harvested at time points ranging from 0 to 48 hours. Serum concentration of SACLAC was measured by mass spectrometry quantification relative to SACLAC standard using the method referenced above.

To assess the efficacy of SACLAC in a transplantable human AML model, two cell line models were used. For the MV4-11 model, 2.5×10⁶ luciferase and yellow fluorescent protein (YFP)-expressing MV4-11 cells in HBSS were introduced into 6- to 8-week-old female NSG mice via tail-vein injections. After 10-12 days, engraftment was confirmed using bioluminescence imaging with the IVIS Lumina LT Series III imaging system (PerkinElmer Inc., Waltham, Mass., United States of America). Mice were equally randomized into two groups of three mice and treated with vehicle or SACLAC at 5 mg/kg/day five times per week for 18 total injections. At the end of the treatment, animals were euthanized and peripheral blood was collected for flow cytometry analysis.

For the U937 model, 1×10⁴ luciferase and tdTomato-expressing U937 cells in HMS were introduced into 6- to 8-week-old female NSG mice via tail-vein injections. After six days, engraftment was confirmed as stated above. Mice were equally randomized into two groups of five mice and treated with vehicle or SACLAC at 5 mg/kg/day five times per week for 15 total injections. Animals were euthanized and bone marrow was collected for flow cytometry analysis.

Red blood cells (RBC) were lysed with RBC Lysis Buffer (#420301, BioLegend, San Diego, Calif., United States of America) followed by cell surface staining with anti-human CD45 antibodies (#304014, clone HI30, BioLegend, San Diego, Calif., United States of America) and 7AAD (#420404, BioLegend, San Diego, Calif., United States of America) to detect viable human white blood cells (WBC) using an LSR II flow cytometer and BD FACS Diva software. Viable cells were identified by gating on 7AAD-negative cells gated from singlets (SSC-A and FSC-W scales), after excluding the debris (SSC-A and FSC-A scales). Percentage of YFP, tdTomato or hCD45 positive cells was determined in FlowJo software (Becton Dickinson, Franklin Lakes, N.J., United States of America). All animal studies were performed with IACUC approval.

### Statistical Analysis

Significance between two treatment groups was determined by the two-tailed unpaired t-test using the GraphPad Prism 7.0 software (GraphPad Software, San Diego, Calif., United States of America). Data sets containing multiple comparison were corrected using the Holm-Sidak method. Experiments containing dose response or time courses were analyzed using one-way ANOVA with Dunnett's post-test to compare each condition to a single control. Cell line studies were repeated for three independent experiments, each with three or more technical replicates unless otherwise stated. One representative experiment is shown with error bars representing standard error of the mean (SEM).

### Example 1

### SACLAC Inhibits AC and Shifts Lipid Levels Toward a Pro-Death Phenotype

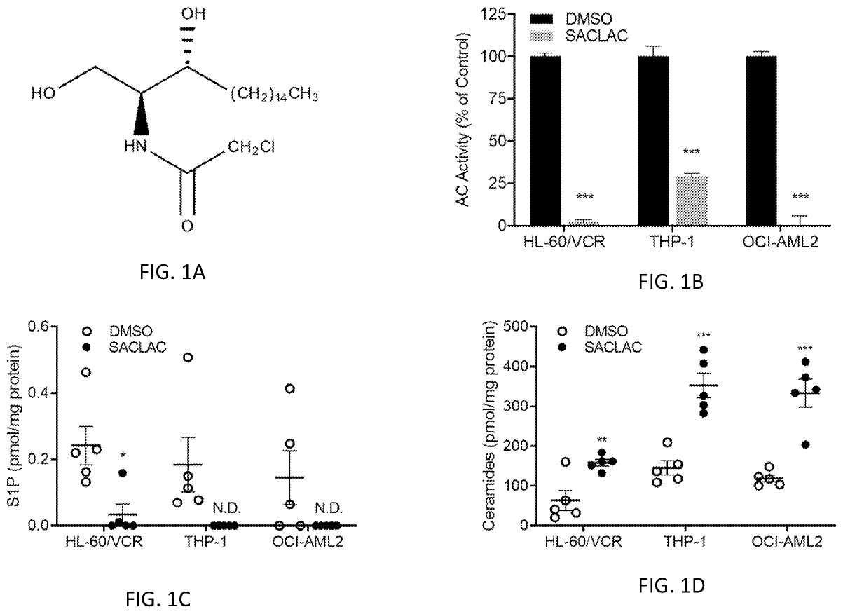

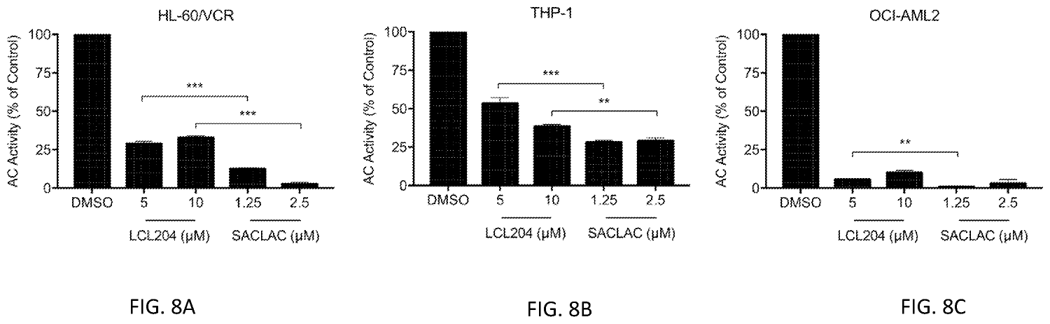

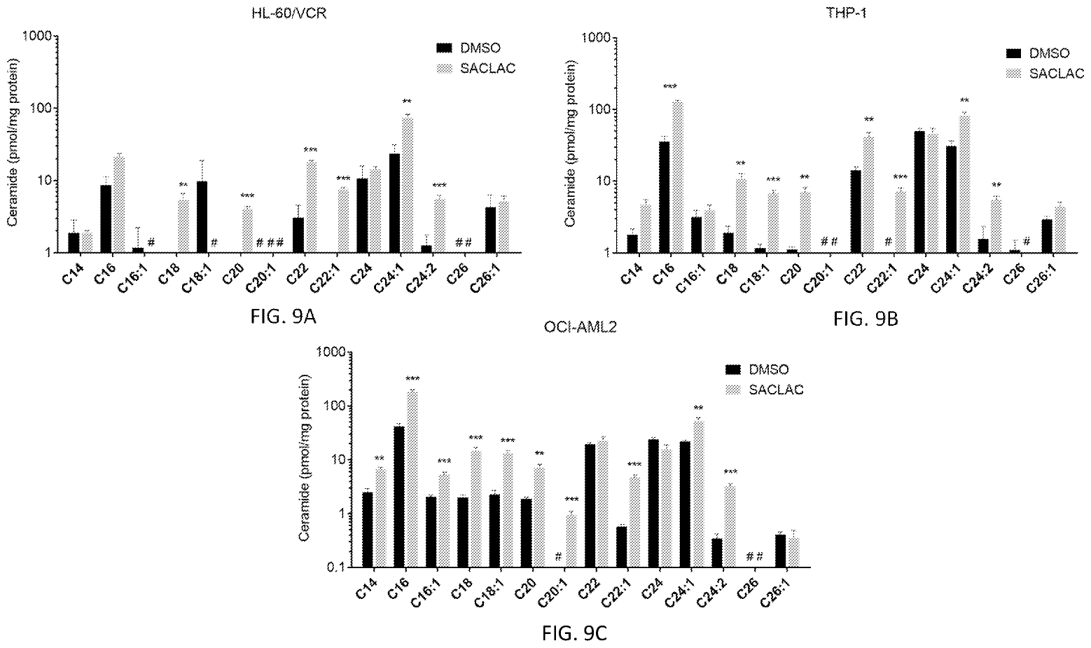

SACLAC is an a-chloroamide ceramide analog (see FIG. 1A) that binds irreversibly to AC by transferring a covalent adduct to the enzyme catalytic site (33). Three human AML cell lines were selected to evaluate SACLAC inhibition of AC activity in AML. The multidrug resistant HL-60/VCR cell line, which overexpresses P-gp (23) was treated alongside THP-1 and OCI-AML2 cell lines with SACLAC (2.5 μM). AC activity of these cell lines was reduced by 98%, 71% and 100%, respectively, with 24-hour treatment. See FIG. 1B. This is superior to LCL204, which requires a four-fold higher dose to achieve similar effects. See FIGS. 8A-8C. Changes in S1P and ceramides were measured under the same conditions. SACLAC reduced S1P content by 87% in HL-60/VCR and reduced S1P levels to below the limit of detection in THP-1 and OCI-AML2 cell lines. See FIG. 1C. Further, these conditions led to a 2- to 3-fold increase in total ceramide production. See FIG. 1D. Most individual ceramide species increased upon SACLAC treatment, with the most pronounced changes being C16, C18, C22:1 and C24:2. See FIGS. 9A-9C. Together these data indicate that SACLAC robustly inhibits AC activity and shifts lipid levels by causing the accumulation of ceramides by blocking the breakdown of ceramide to sphingosine and further conversion to SIP, thereby increasing pro-death ceramides and decreasing pro-survival SIP.

### Example 2

### SACLAC Reduces Viability and Colony Formation

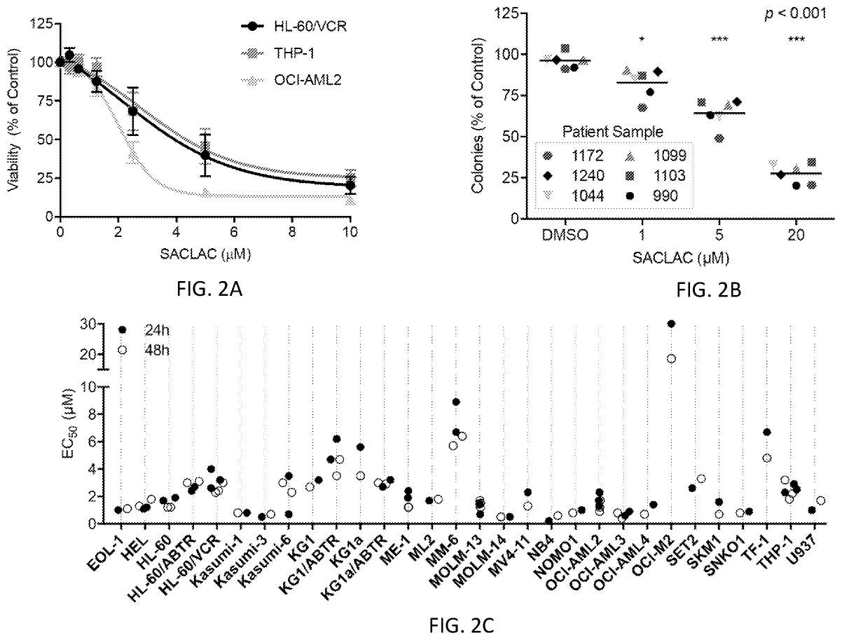

To determine if these shifts in lipid content were functionally significant and universally observed in human AML cell lines and patient samples, the same three representative cell lines were treated with SACLAC and cell viability was determined by MTS assay. HL-60/VCR, THP-1 and OCI-AML2, had EC₅₀ values in the low-micromolar range (3.3, 2.6 and 1.8 μM, respectively) with 24-hour treatment. See FIG. 2A. To determine if this effect translated to AML patient cells, colony formation was analyzed in a panel of six primary patient samples. These six primary patient samples showed a reduced ability to form colonies in the presence of increasing doses of SACLAC. On average, colony formation decreased 32% with 5 μM treatment and 69% with 20 μM treatment. See FIG. 2B. Since AML is a heterogeneous disease, 30 human AML cell lines were assayed by MTS assay at 24 and 48-hour time points. Remarkably, 25 of 30 cell lines had ECso values below 5 μM, with an average EC₅₀ of 3.2 μM. See FIG. 2C. The outlier cell line OCI-M2 could be an interesting cell line to investigate further in the context of sphingolipid metabolism, AC and mechanisms of SACLAC action and/or resistance.

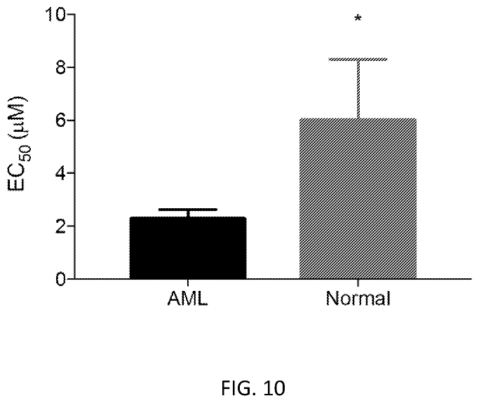

SACLAC treatment for 24 hours slightly reduced normal cell viability. PBMCs (n=6) had an average EC₅₀ of 7.4 μM while CD34⁺ (n=4) cells averaged 4.0 μM. See FIG. 10. These data show that, although there is variability in sensitivity, SACLAC is broadly toxic to AML cells with lesser toxicity in normal cells.

### Example 3

### SACLAC Induces Apoptosis and Loss of Mitochondrial Membrane Potential

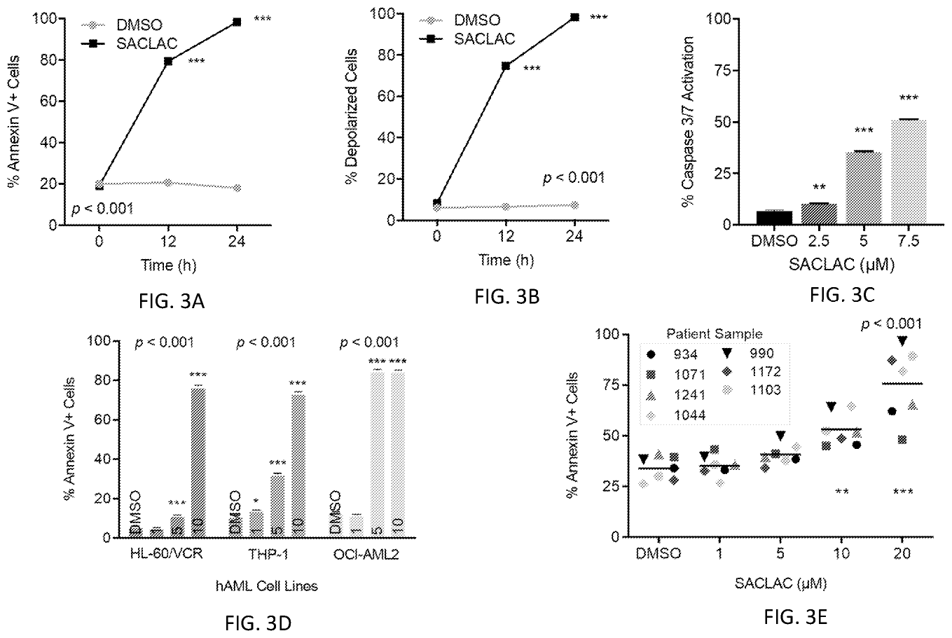

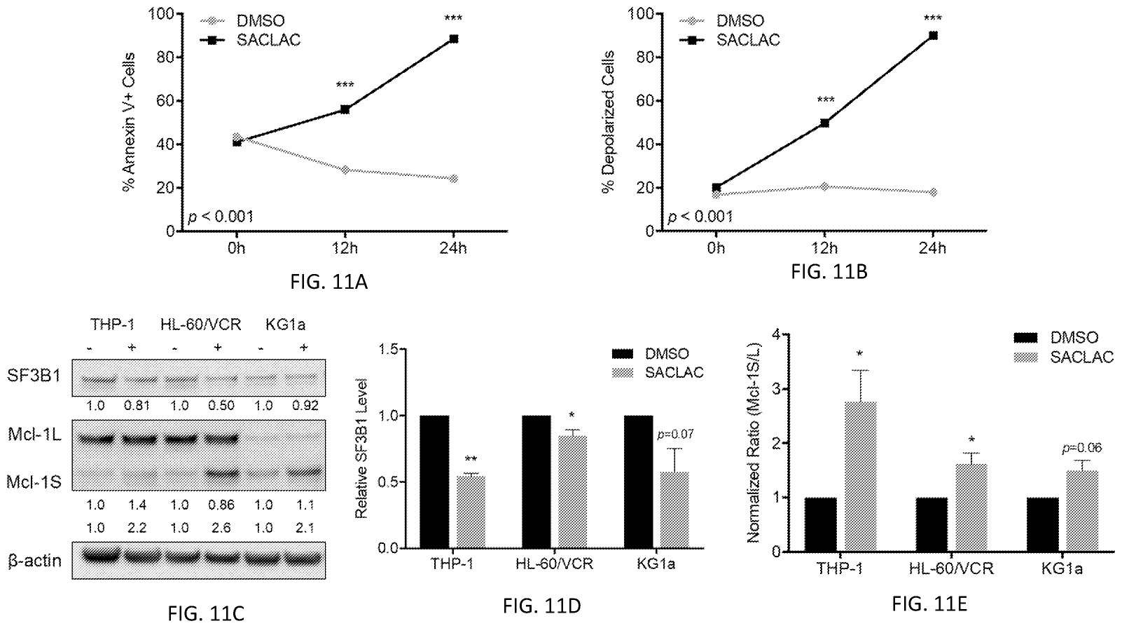

After confirming that SACLAC treatment reduced cell viability, the mechanism of cell death was examined. First, apoptosis induction was evaluated over time in OCI-AML2 cells treated with SACLAC (5 μM). See FIG. 3A. Apoptosis was induced as early as 12 hours (79% apoptosis) with nearly all cells undergoing apoptosis at 24 hours. Loss of mitochondrial membrane potential (see FIG. 3B) occurred in parallel with positive staining for the apoptosis marker annexin V. See FIG. 3A. These patterns were also observed in an additional cell line, THP-1. See FIGS. 11A and 11B. Additionally, dose-dependent caspase activation was observed with SACLAC treatment. See FIG. 3C. Three human AML cell lines exhibited dose-dependent induction of Annexin V staining with 48-hour SACLAC treatment, albeit with varying sensitivity. See FIG. 3D. AML patient samples exhibited similar results with more than half the samples reaching 80% apoptosis or greater with SACLAC treatment. See FIG. 3E. These data suggest that apoptosis is the predominant mechanism for SACLAC-induced cell death in AML cells.

### Example 4

### SACLAC Alters the Ratio of Pro-survival to Pro-Apoptotic Mcl-1 Isoforms

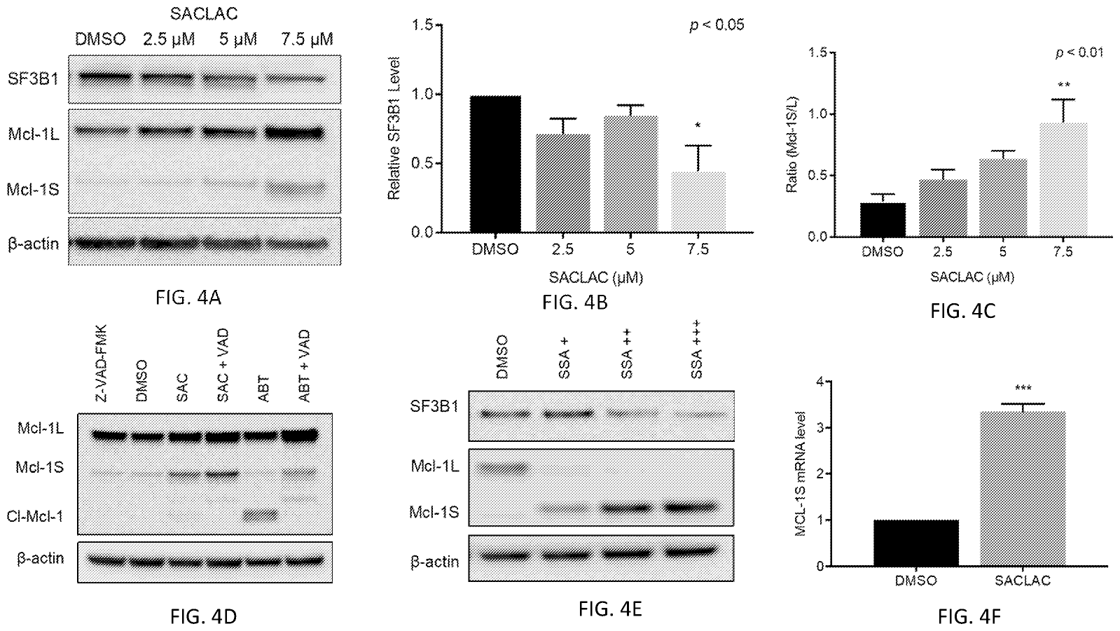

Next, mechanisms that mediate apoptosis induction in SACLAC-treated OCI-AML2 cells after 24-hour exposure to increasing doses of SACLAC were studied. Since Mcl-1 has been implicated in AML pathogenesis and specifically in response to sphingolipid modulation (22), changes in Mcl-1 with SACLAC treatment were examined. Most notably, SACLAC induced a 10-fold upregulation of the Mcl-1S isoform, which translated to a 3-fold increase in the ratio of pro-apoptotic Mcl-1S to pro-survival Mcl-1L. See FIGS. 4A and 4C. Since changes in Mcl-1S were observed, splicing factors known to regulate Mcl-1 were also examined. SACLAC treatment reduced SF3B1 protein expression (see FIGS. 4A and 4B), suggesting that spliceosome assembly and exon inclusion is disrupted (40). Another splicing factor, SRSF1, was not affected. Because Mcl-1 can also be cleaved by caspases to generate a smaller protein fragment, ABT-737-treated cells were used as a positive control for caspase-mediated Mcl-1 cleavage (41). ABT-737 treatment resulted in the expected smaller fragment of approximately 22 kDa that was abrogated by co-treatment with caspase inhibitor Z-VAD-FMK. However, SACLAC treatment led to the appearance of the larger Mcl-1S at about 32 kDa, which is the result of alternative mRNA splicing and was not blocked by caspase inhibitor treatment. See FIG. 4D. SF3B1 inhibition with Spliceostatin A (SSA) resulted in a dose-dependent increase in Mcl-1S (see FIG. 4E), which confirms SF3B1's role in Mcl-1 splicing in OCI-AML2 cells. Mcl-1 exon 2 exclusion was confirmed using RT-qPCR with primers specific for the Mcl-1S isoform. Increased MCL-1S transcript was observed in OCI-AML2 cells treated with 5 μM SACLAC. See FIG. 4F. In order to determine if this mechanism is conserved across multiple AML cell lines, changes in protein signaling were examined after treating THP-1, HL60-VCR and KG1 a cell lines with SACLAC for 48 hours. Similar to OCI-AML2 cells, SACLAC reduced SF3B1 levels and increased the pro-apoptotic ratio of Mcl-1S/L in all three cell lines. See FIGS. 11C-11E. Without being bound to any one theory, these observations implicate SF3B1 and Mcl-1S in SACLAC-mediated intrinsic apoptosis of AML cells.

### Example 5

### C16 Ceramide Treatment and AC Knockdown Reduce SF3B1 and Increase MCL-1S/L Ratio

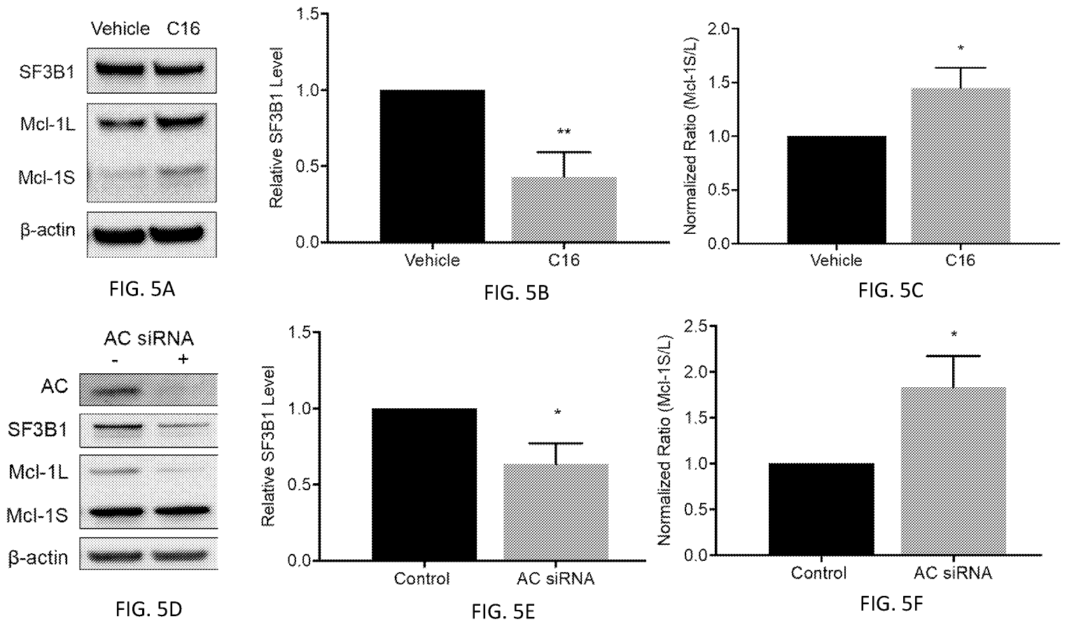

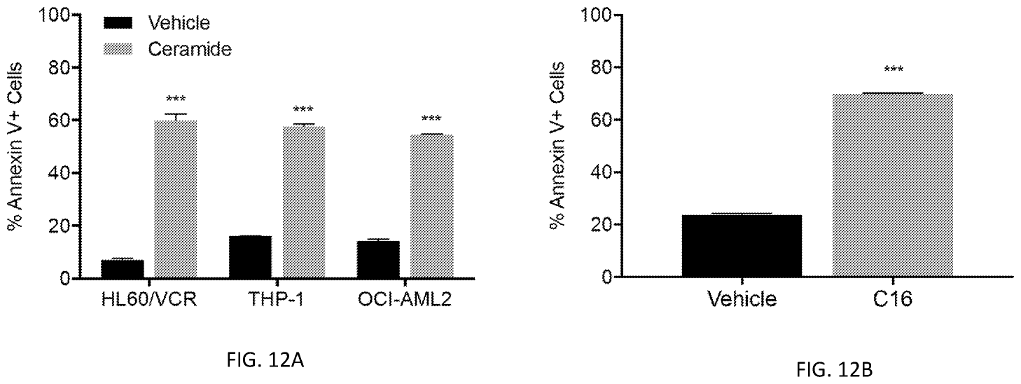

In order to determine if ceramide is upstream of this mechanistic observation, three human AML cell lines were treated with a 100 μg/ml (˜150 μM) mixture of ceramides, predominantly C18 species and longer. The ceramide mixture induced 60% apoptosis with 48-hour treatment. See FIG. 12A. Treatment with C16 ceramide (20 μM) induced similar levels of apoptosis. See FIG. 12B. Further, treatment with C16 ceramide induced similar mechanistic effects as SACLAC, with a reduction in SF3B1 and an increase in the ratio of Mcl-1S to Mcl-1L. See FIGS. 5A-5C. These data demonstrate that exogenous supplementation with ceramide species, which were shown to increase in response to SACLAC treatment (see FIGS. 9A-9C), is sufficient to induce apoptosis and alternative Mcl-1 splicing in AML. To determine if SACLAC effects on AML cells are dependent on inhibition of AC activity, AC protein was knocked down using siRNA. Treatment of KGla cells with siRNA targeting AC reduced AC protein by 76% at 48 hours post-transfection. See FIG. 5D. At this time point, SF3B1 was reduced 37% and the ratio of Mcl-1S to Mcl-1L increased by 83%. See FIGS. 5E and 5F.

### Example 6

### Altering Expression of MCL-1 and SF3B1 Attenuates Induction of Apoptosis in SACLAC-Treated Cells

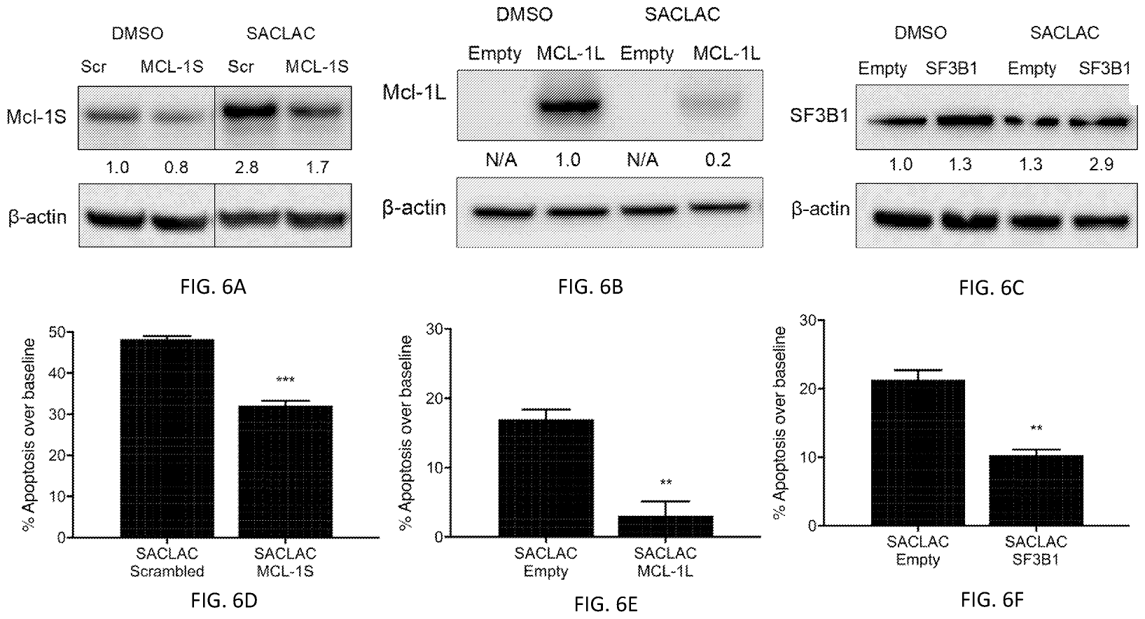

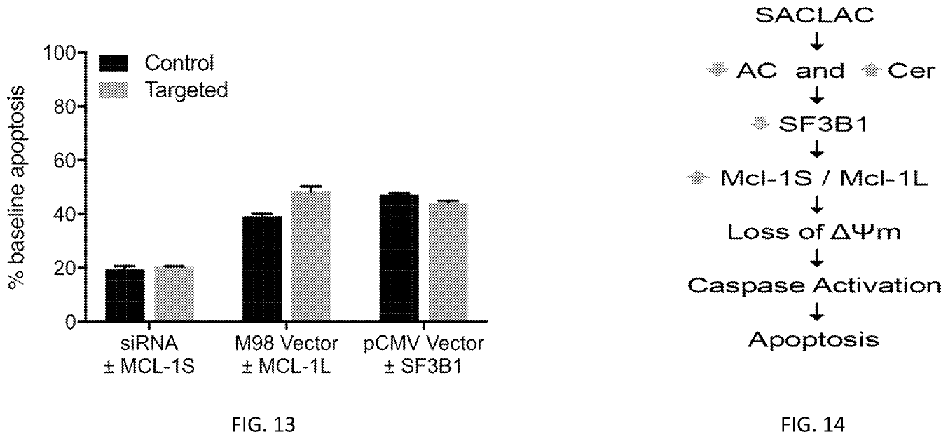

The role of Mcl-1S in the mechanism of SACLAC-mediated cell death was investigated further. KG1a cells were electroporated with a siRNA (#CTM-481502, Dharmacon, Lafayette, Colo., United States of America) designed to target the junction of exons 1 and 3, which is present only in the MCL-1S transcript, and treated with DMSO or SACLAC. Presence of short isoform-specific siRNA attenuated the induction of Mcl-1S protein expression upon SACLAC treatment. See FIG. 6A. Further, apoptosis induction was reduced by 36% in SACLAC-treated Mcl-1S knockdown cells. See FIG. 6D. Next, cDNA expression vectors were used to overexpress MCL-1L (see FIG. 6B) or SF3B1 (see FIG. 6C) in KGla cells. Each of these conditions led to a partial rescue of apoptosis in SACLAC-treated cells. See FIGS. 6E and 6F. Electroporation of expression vectors led to increased baseline apoptosis in KGla cells. See FIG. 13. Therefore, the magnitude of SACLAC induced apoptosis after baseline subtraction is reduced in FIGS. 6E and 6F relative to FIG. 6D. In summary, SACLAC treatment inhibits AC, which leads to increased ceramide and decreased SF3B1. As ceramide accumulates, alternative splicing of Mcl-1 results in Mcl-1S accumulation that facilitates mitochondrial membrane depolarization. With loss of mitochondrial membrane potential (ΔΨm), pro-apoptotic signals are released to activate caspases and induce apoptosis. See FIG. 14.

### Example 7

### SACLAC Reduces Leukemic Burden in NSG Mouse Models of AML

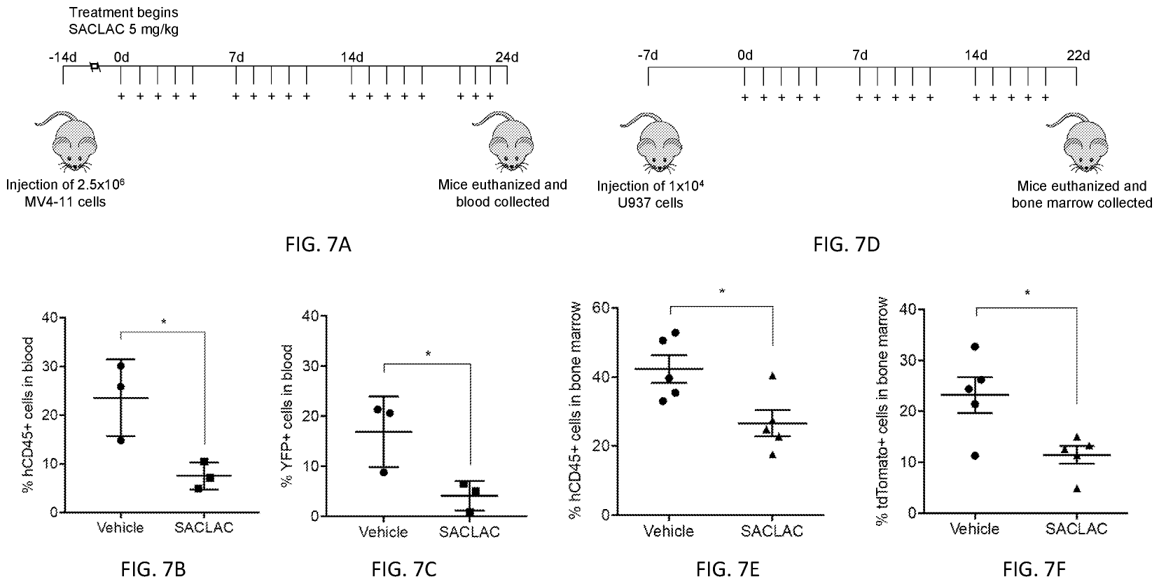

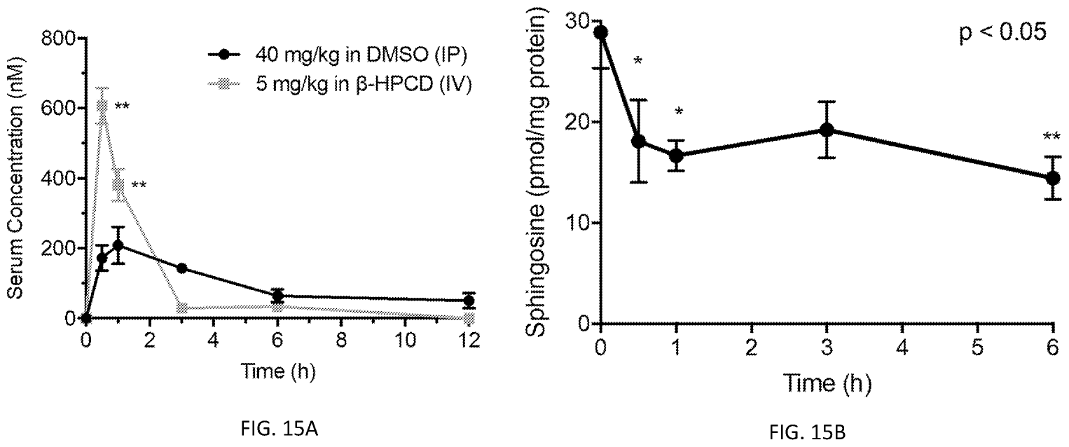

NOD-scid IL2Rgammana (NSG) immunodeficient mice were engrafted with human MV4-11 AML cells and then treated with 5 mg/kg SACLAC five times per week by tail-vein injection. See FIG. 7A. This dose and route of administration were chosen based upon studies indicating that IV delivery yields a higher maximal serum concentration than IP delivery. See FIG. 15A. SACLAC exhibited a short half-life in the blood (see FIG. 15A), yet levels of the sphingosine product were reduced in the liver for at least 6 hours after treatment. See FIG. 15B. After 18 injections, circulating leukemic cells in the blood were counted using flow cytometry. SACLAC treatment resulted in a significant (˜75%) decrease in leukemic burden. See FIGS. 7B and 7C. No overt toxicity was observed and SACLAC treated animals exhibited weight gain. A second, more aggressive model using U937 AML cells (see FIG. 7D) exhibited up to 50% reduction in leukemic cells in the bone marrow of SACLAC-treated mice. See FIGS. 7E and 7F. These data in two preclinical human AML xenograft models highlight the potential therapeutic efficacy of SACLAC in AML.

### Example 8

### Discussion of Examples 1-7

The AC inhibitor SACLAC kills cells via intrinsic apoptosis mediated through a signaling pathway that includes ceramide and Mcl-1S. SACLAC binds directly to the catalytic site of AC. SACLAC is broadly cytotoxic across AML cell lines and patient samples. SACLAC treatment reduces leukemic burden in mice, even at nanomolar serum concentrations. Moreover, therapeutic effects were observed in murine xenograft models despite limited ability to deliver high doses of the compound. These in vivo models affirm that SACLAC is non-toxic at the administered therapeutic dose.

The present studies identify a novel association between AC inhibition and alternative splicing in AML. Mcl-1 protein is clearly associated with survival of AML cells (24). Mcl-1 and sphingolipids have been linked by previous work connecting Mcl-1 with AC (22), S1P (45), and ceramide signaling (46). However, these “traditional” relationships focused on changes in the full length Mcl-1 protein (Mcl-1L). In contrast, the present studies highlight a unique mechanism whereby altered splicing leads to a pro-apoptotic Mcl-1 ratio. This role for Mcl-1S in SACLAC-mediated cell death is reinforced by a partial rescue of viability with knockdown of Mcl-1S as well as overexpression of Mcl-1 or SF3B1. See FIGS. 6B and 6F. These results suggest that alternative splicing of Mcl-1 cooperates with and compliments other apoptotic contributors, such as ceramide accumulation, S1P depletion and diminished Mcl-1L, to enhance SACLAC-mediated cell death.

The relationship between SF3B1 and Mcl-1 has been established (28), but the molecular mechanism whereby elevated ceramide and/or SACLAC levels lead to SF3B1 reduction is unclear. Without being bound to any one theory, ceramides appear to regulate SF3B1 expression through activation of protein phosphatase 1 (PP1), which is known to bind the RNA recognition motif of several splicing factors (47).

Reduced SF3B1 levels and increased Mcl-1S/L ratios were observed consistently upon AC knockdown, SACLAC treatment and ceramide supplementation. See FIGS. 4A-4F and 5A-5F). The AC knockdown results differed slightly in that loss of Mcl-1L was a major factor in the 2-fold increase of Mcl-1S to Mcl-1L (see FIG. 5D) as opposed to primarily an increase in Mcl-1S in the other models. Without being bound to any one theory, this difference is likely associated with variation in temporal dynamics and sphingolipid subcellular distribution associated with different types of manipulation. For example, AC activity loss can be more rapid and complete with inhibitor treatment relative to siRNA-mediated knockdown. Another likely contributor is the abundance of Mcl-1S and L isoforms in KG1 a cells relative to other AML cell lines. See FIG. 11C. Loss of Mcl-1L can also occur upon LCL-204 treatment and AC knockdown in multiple human AML cell lines (22). LCL204 is known to induce lysosomal disruption that likely contributes to the proteasomal degradation of Mcl-1 observed in our previous studies, which did not investigate levels of Mcl-1S (22). These varying Mcl-1 alterations can relate to the diverse and complex regulatory mechanisms that control Mcl-1 transcription, stability and localization (22,48). Indeed, a reduction in Mcl-1L levels was also detected with the exogenous overexpression of a Mcl-1L/EGFP. Expanding the understanding of temporal and spatial relationships when utilizing sphingolipid modulators or sphingolipids themselves, as well as the downstream effects on Mcl-1, can enhance future therapeutic targeting of sphingolipid metabolism.

The observed changes in SF3B1 and Mcl-1 are of interest since a subset of the common somatic mutations in AML involve genes that regulate RNA binding and spliceosome assembly (49). Interestingly, myelodysplastic syndrome (MDS), which sometimes precedes AML, exhibits spliceosome mutations in about 50% of cases and responds noticeably well to spliceosomal inhibition (32). Additional clinical relevance of the present studies is highlighted by Mcl-1's role in resistance to Bcl-2 inhibitor venetoclax (ABT-199) (25,50). Venetoclax was recently granted breakthrough status for the treatment of newly diagnosed AML, in combination with hypomethylating agents or low dose cytarabine (also known as AraC) (51,52). Results regarding the relative efficacy of SACLAC combinatorial treatments, including those involving the combination of SACLAC and venetoclax are described further below in Example 10.

The delivery of SACLAC can be improved changing administration route and vehicle (see FIG. 15A), but only achieved peak serum SACLAC levels of less than 1 μM. The control and SACLAC-treated mice were of similar health at termination of the study. SACLAC-treated mice did not lose weight over the course of the study. Nonetheless, significant differences in leukemic burden was observed at the cellular level. Identifying improved solvents, alternative formulations or structural derivatives of SACLAC can improve therapeutic efficacy. Alternative formulations are explored, for example, in Example 9, below.

The presently disclosed findings are particularly of note because of AML patient heterogeneity (5). While mutation-targeted therapeutics are best suited for specific patient subpopulations, SACLAC appears to exploit a common biochemical dependence across the vast majority of AML cell lines and patient samples. The screen of 30 AML cell lines represents the largest panel of AC inhibitor efficacy studies in AML to date. Further, the present studies elucidate the mechanism of SACLAC cytotoxicity in AML cells, which can inform future efforts to design combination therapies or combat potential resistance.

The present data support the involvement of AC, ceramide, SF3B1 and Mcl-1 in SACLAC-mediated apoptosis. Together, the present studies demonstrate that ceramide accumulation leading to SF3B1 reduction and Mcl-1S induction is at least one mechanism of action for the exemplary AC inhibitor SACLAC.

### Example 9

### Formulation Studies

SACLAC exhibits low solubility in vehicles for i.v. drug delivery and suboptimal uptake following i.p. injection. Accordingly, additional formulations for SACLAC were studied to maximize the in vivo delivery of SACLAC.

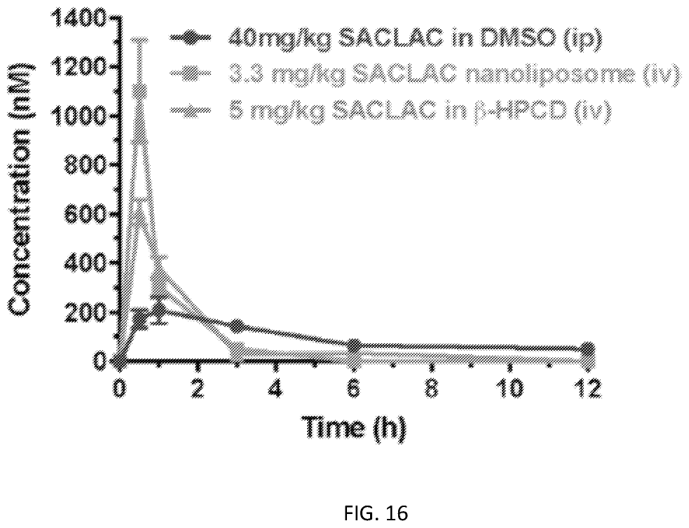

More particularly, SACLAC was dissolved in 2-hydroxypropyl-β-cyclodextrin (β-HPCD) (53). In addition, a SACLAC nanoliposome was prepared. Following the injection of free SACLAC in DMSO (i.p.), β-HPCD (i.v.) or the SACLAC nanoliposome (nanoSACLAC, i.v.) low serum levels were measured upon i.p. delivery, with improvement following i.v. delivery and the highest levels with nanoSACLAC. See FIG. 16. The maximum serum level (˜1 μM) was near the EC₅₀ in hAML cell lines, although SACLAC was rapidly cleared in all treatment groups.

Initial encapsulation of compound was done in neutral nanoliposomes (54, 55), via technology with the capability to optimize encapsulation efficiency, stability, and in vivo behavior by fine-tuning the levels of cholesterol and PEG in the nanoliposome. Second-generation nanoliposomes with increased cholesterol content exhibited improved efficacy in vitro. The compounds will also be loaded in anionic ceramide liposomes that contain dihexadecylphosphate, cholesterol, and a lower molar percentage of bioconjugated PEG to facilitate receptor-mediated endocytosis.

Finally, the compounds can be encapsulated in PEO-PDLLA diblock copolymers through flash nanoprecipitation (56, 57). Initial polymer formulations were potent in three hAML cell lines (580 to 780 nM EC₅₀), which represents 1.4 to 3.1-fold improvement over free SACLAC. In addition, SACLAC was encapsulated mPEGPLGA polymer nanoparticles by bulk nanoprecipitation or solvent displacement methodologies. PEG chain length of the polymer (1K to 10K) and the lactide (LA): glycolide (GA) ratio (10:90: to 90:10) have been created, e.g., with a 5K PEG and 50:50 LA:GA ratio. SACLAC was dissolved in acetonitrile (ACN) at 1 mg/mL concentration and the m-PEG PLGA polymer was dissolved in ACN at 10 mg/mL concentration.

### Example 10

### Combination Therapies

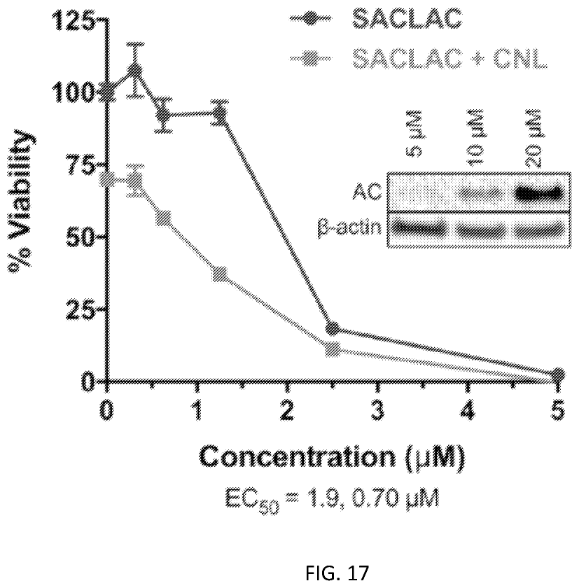

Upregulated AC expression has been demonstrated in C6-ceramide nanoliposome (CNL)-treated hAML cells. In addition, enhanced killing was demonstrated when CNL was combined with SACLAC. See FIG. 17. Thus, without being bound to any one theory, it appears that AC is induced by CNL, and AC inhibitors yield synergistic killing together with CNL.

Venetoclax (also known as ABT-199) sensitive (MV4-11, MOLM-13, OCL-AML2) and venetoclax resistant (MM-6, THP-1, HL-60/VCR, OCI-AML3) hAML cell lines have been identified that exhibit >100-fold difference in sensitivity. In addition, ABT-737-resistant (ABTR) variants of HL-60, KG1, and GK1 a cells have been developed (39) and have demonstrated that resistance is associated with Mcl-1 upregulation. The HL-60/ABTR cell line also exhibits cross-resistance to venetoclax. Venetoclax sensitive cell lines can be co-treated with SACLAC (0.3 to 2.5 μM) and venetoclax (1 to 32 nM). Venetoclax resistant cells can be co-treated with SACLAC and higher concentrations of venetoclax (0.2 to 5 μM). Cell viability can be determined by MTS assay 24 hours after treatment. CompuSyn software (ComboSyn Inc., Paramus, N.J., United States of America) can be used to determine synergy. A combination index (CI) of >1, 1, and <1 indicates antagonism, additive effect, and synergism, respectively, with increasing synergism indicated by values approaching zero.

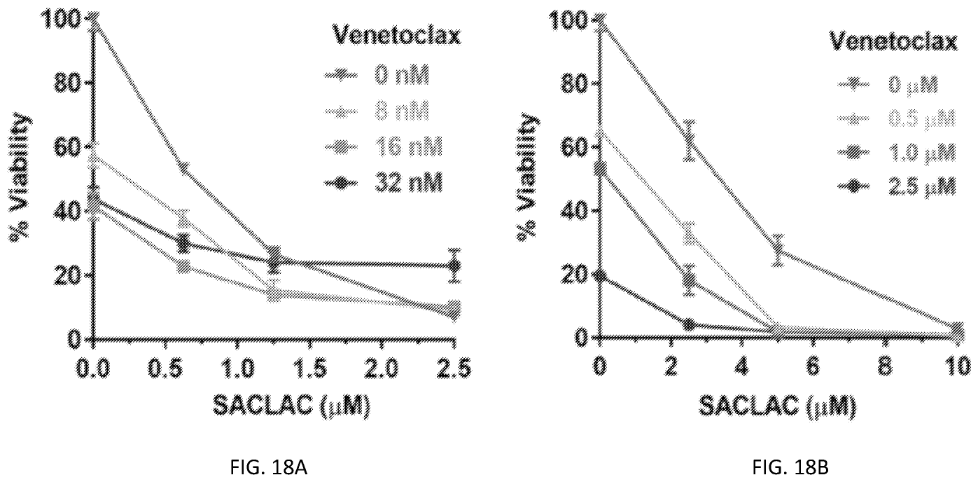

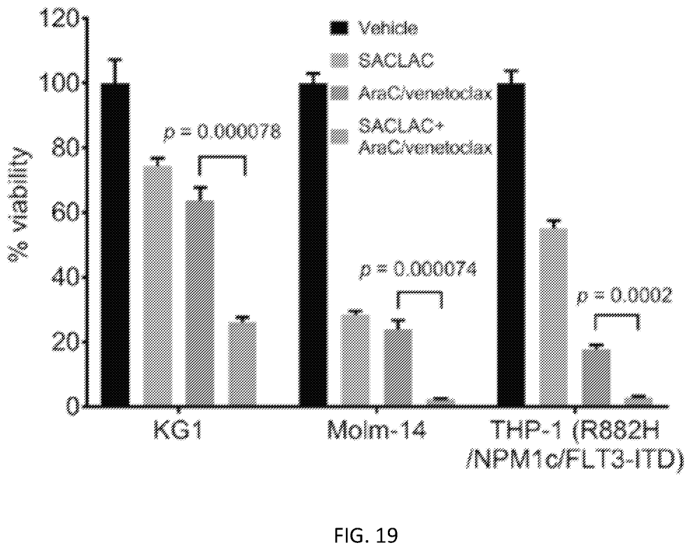

The SACLAC and venetoclax combination exhibited synergistic killing in MV4-11 and MM-6 hAML cells that are intrinsically sensitive or resistant, respectively, to venetoclax. See FIGS. 18A and 18B. The combination exhibited an average CI of 0.58 in MM-6 cells. The efficacy of SACLAC was also tested in combination with AraC/venetoclax to evaluate synergistic activity with this therapeutic approach, which exhibits clinical efficacy in AML patients non-eligible for intensive therapy (58, 59). Studies in three AML cell lines of different mutational status (including those expressing mutant DNMT3A, NPM1 and FLT3, far right) indicate promising combinatorial efficacy of SACLAC in this context. See FIG. 19.

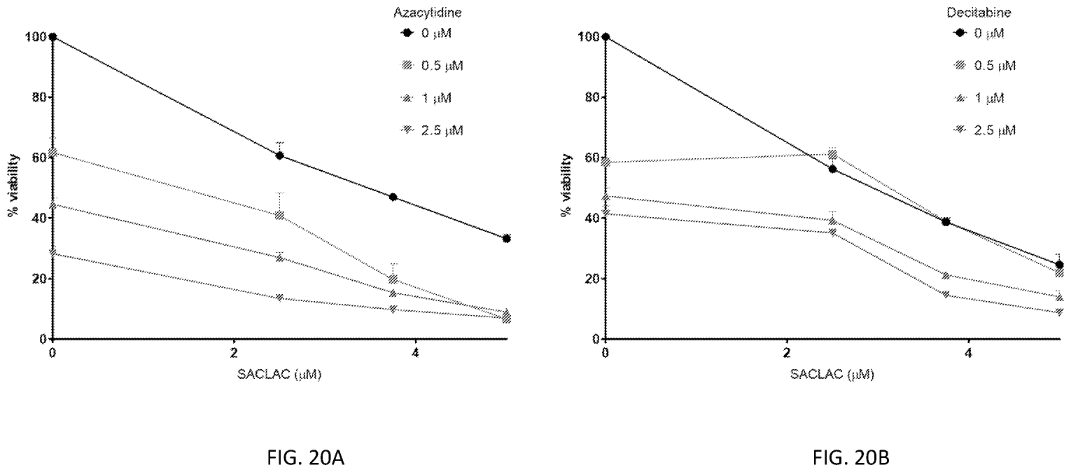

In addition, SACLAC was tested in combination with the hypomethylating agents (HMA) azacytidine and decitabine. HMAs are primarily utilized in AML patient populations that are ineligible for or unable to tolerate intensive chemotherapy (60) and, more recently, as maintenance following intensive chemotherapy (61). HMAs have also shown enhanced efficacy when combined with venetoclax in previously untreated as well as relapsed AML (62, 63). Viability assays in the drug resistant HL-60/VCR human AML cell line, which is known to overexpress P-glycoprotein (P-gp), demonstrated synergistic efficacy upon combinatorial treatment with SACLAC plus azacytidine (lowest CI value of 0.34; see FIG. 20A) as well as SACLAC plus decitabine (lowest CI value of 0.53) See FIG. 20B.

### REFERENCES

The references listed below as well as all references cited in the specification are incorporated herein by reference to the extent that they supplement, explain, provide a background for or teach methodology, techniques and/or compositions employed herein. All cited patents and publications referred to in this application are herein expressly incorporated by reference.
1. Noone A, Howlader N, Krapcho M, Miller D, Brest A, Yu M, et al. SEER Cancer Statistics Review, 1975-2015 [Internet]. Natl. Cancer Inst. [cited 2018 Sep 4]. Available from: https://seer.cancer.gov/csr/1975_2015/2.
2. Klepin H D, Balducci L. Acute Myelogenous Leukemia in Older Adults. The Oncologist. 2009; 14:222-32.
3. Dombret H, Gardin C. An update of current treatments for adult acute myeloid leukemia. Blood. 2016;127:53-61.
4. Hatzimichael E, Georgiou G, Benetatos L, Briasoulis E. Gene mutations and molecularly targeted therapies in acute myeloid leukemia. Am J Blood Res. 2013; 3:29-51.
5. Martelli M P, Sportoletti P, Tiacci E, Martelli M F, Falini B. Mutational landscape of AML with normal cytogenetics: biological and clinical implications. Blood Rev. 2013; 27:13-22.
6. Tyner J W, Tognon C E, Bottomly D, Wilmot B, Kurtz S E, Savage S L, et al. Functional genomic landscape of acute myeloid leukaemia. Nature. 2018; 562:526-31.
7. Cancer Genome Atlas Research Network, Ley T J, Miller C, Ding L, Raphael B J, Mungall A J, et al. Genomic and epigenomic landscapes of adult de novo acute myeloid leukemia. N Engl J Med. 2013; 368:2059-74.
8. Cortes J, Perl A E, Döhner H, Kantarjian H, Martinelli G, Kovacsovics T, et al. Quizartinib, an FLT3 inhibitor, as monotherapy in patients with relapsed or refractory acute myeloid leukaemia: an open-label, multicentre, single-arm, phase 2 trial. Lancet Oncol. 2018; 19:889-903.
9. Wiese M, Daver N. Unmet clinical needs and economic burden of disease in the treatment landscape of acute myeloid leukemia. Am J Manag Care. 2018; 24:S347-55.
10. Hong W-J, Medeiros B C. Unfavorable-risk cytogenetics in acute myeloid leukemia. Expert Rev Hematol. 2011; 4:173-84.
11. Papaemmanuil E, Gerstung M, Bullinger L, Gaidzik V I, Paschka P, Roberts N D, et al. Genomic Classification and Prognosis in Acute Myeloid Leukemia. N Engl J Med. 2016; 374:2209-21.
12. Buckley S A, Othus M, Estey E H, Walter R B. The treatment-related mortality score is associated with non-fatal adverse events following intensive AML induction chemotherapy. Blood Cancer J. 2015; 5:e276.
13. Burrell R A, Swanton C. Tumour heterogeneity and the evolution of polyclonal drug resistance. Mol Oncol. 2014; 8:1095-111.
14. Ryland L K, Fox T E, Liu X, Loughran TP, Kester M. Dysregulation of sphingolipid metabolism in cancer. Cancer Biol Ther. 2011; 11:138-49.
15. Takabe K, Spiegel S. Export of sphingosine-1-phosphate and cancer progression. J Lipid Res. 2014; 55:1839-46.
16. Morad S A F, Cabot M C. Ceramide-orchestrated signalling in cancer cells. Nat Rev Cancer. 2013; 13:51-65.
17. Mao C, Obeid L M. Ceramidases: regulators of cellular responses mediated by ceramide, sphingosine, and sphingosine-1-phosphate. Biochim Biophys Acta. 2008;1781:424-34.
18. Camacho L, Meca-Cortés O, Abad J L, Garcia S, Rubio N, Diaz A, et al. Acid ceramidase as a therapeutic target in metastatic prostate cancer. J Lipid Res. 2013;54:1207-20.
19. Leclerc J, Garandeau D, Pandiani C, Gaudel C, Bille K, Nottet N, et al. Lysosomal acid ceramidase ASAH1 controls the transition between invasive and proliferative phenotype in melanoma cells. Oncogene. 2019;38.
20. Flowers M, Fabriás G, Delgado A, Casas J, Abad J L, Cabot MC. C6-ceramide and targeted inhibition of acid ceramidase induce synergistic decreases in breast cancer cell growth. Breast Cancer Res Treat. 2012;1 33:447-58.
21. Nguyen H, Awad A, Shabani S, Doan N, Nguyen H S, Awad A J, et al. Molecular Targeting of Acid Ceramidase in Glioblastoma: A Review of Its Role, Potential Treatment, and Challenges. Pharmaceutics. 2018; 10:45.
22. Tan S-F, Liu X, Fox T E, Barth B M, Sharma A, Turner S D, et al. Acid ceramidase is upregulated in AML and represents a novel therapeutic target. Oncotarget. 2016; 7:83208-22.
23. Tan S-F, Dunton W, Liu X, Fox T E, Morad S A F, Desai D, et al. Acid ceramidase promotes drug resistance in acute myeloid leukemia through NF-kB-dependent P-glycoprotein upregulation. J Lipid Res. 2019;
24. Glaser S P, Lee E F, Trounson E, Bouillet P, Wei A, Fairlie W D, et al. Anti-apoptotic Mcl-1 is essential for the development and sustained growth of acute myeloid leukemia. Genes Dev. 2012; 26:120-5.
25. Luedtke D A, Niu X, Pan Y, Zhao J, Liu S, Edwards H, et al. Inhibition of Mcl-1 enhances cell death induced by the Bcl-2-selective inhibitor ABT-199 in acute myeloid leukemia cells. Signal Transduct Target Ther. 2017; 2:17012.
26. Teh T-C, Nguyen N-Y, Moujalled D M, Segal D, Pomilio G, Rijal S, et al. Enhancing venetoclax activity in acute myeloid leukemia by co-targeting MCL1. Leukemia. 2018; 32:303-12.
27. Aldoss I, Yang D, Aribi A, Ali H, Sandhu K, Al Malki M M, et al. Efficacy of the combination of venetoclax and hypomethylating agents in relapsed/refractory acute myeloid leukemia. Haematologica. 2018; 103: e404-7.
28. Gao Y, Koide K. Chemical perturbation of Mcl-1 pre-mRNA splicing to induce apoptosis in cancer cells. ACS Chem Biol. 2013; 8:895-900.
29. Morciano G, Giorgi C, Balestra D, Marchi S, Perrone D, Pinotti M, et al. Mcl-1 involvement in mitochondrial dynamics is associated with apoptotic cell death. Mol Biol Cell. 2016; 27:20-34.
30. Bae J, Leo C P, Hsu S Y, Hsueh A J W. MCL-1S, a Splicing Variant of the Antiapoptotic BCL-2 Family Member MCL-1, Encodes a Proapoptotic Protein Possessing Only the BH3 Domain. J Biol Chem. 2000; 275:25255-61.
31. Chittenden T. BH3 domains: intracellular death-ligands critical for initiating apoptosis. Cancer Cell. 2002; 2:165-6.
32. Lee SC-W, Abdel-Wahab O. Therapeutic targeting of splicing in cancer. Nat Med. 2016; 22:976-86.
33. Ordóñez Y F, Abad J L, Asseri M, Casas J, Garcia V, Casasampere M, et al. Activity-based imaging of acid ceramidase. J Am Chem Soc. 2019;
34. Gouazé-Andersson V, Flowers M, Karimi R, Fabriàs G, Delgado A, Casas J, et al. Inhibition of acid ceramidase by a 2-substituted aminoethanol amide synergistically sensitizes prostate cancer cells to N-(4-hydroxyphenyl) retinam . . . —PubMed—NCBI. The Prostate. 2011; 71:1064-73.
35. Daniele S, Taliani S, Da Pozzo E, Giacomelli C, Costa B, Trincavelli M L, et al. Apoptosis Therapy in Cancer: The First Single-molecule Co-activating p53 and the Translocator Protein in Glioblastoma. Sci Rep. 2014; 4.
36. Ru Q, Li W, Xiong Q, Chen L, Tian X, Li C-Y. Voltage-gated potassium channel blocker 4-aminopyridine induces glioma cell apoptosis by reducing expression of microRNA-10b-5p. Mol Biol Cell. 2018; 29:1125-36.
37. Alsafadi S, Houy A, Battistella A, Popova T, Wassef M, Henry E, et al. Cancer-associated SF3B1 mutations affect alternative splicing by promoting alternative branchpoint usage. Nat Commun. 2016; 7:10615.
38. Crews L A, Balaian L, Delos Santos N P, Leu H S, Court A C, Lazzari E, et al. RNA Splicing Modulation Selectively Impairs Leukemia Stem Cell Maintenance in Secondary Human AML. Cell Stem Cell. 2016; 19:599-612.
39. Doi K, Liu Q, Gowda K, Barth B M, Claxton D, Amin S, et al. Maritoclax induces apoptosis in acute myeloid leukemia cells with elevated Mcl-1 expression. Cancer Biol Ther. 2014; 15:1077-86.
40. Dolatshad H, Pellagatti A, Fernandez-Mercado M, Yip B H, Malcovati L, Attwood M, et al. Disruption of SF3B1 results in deregulated expression and splicing of key genes and pathways in myelodysplastic syndrome hematopoietic stem and progenitor cells. Leukemia. 2015; 29:1798.
41. Ryu Y, Hall C P, Reynolds C P, Kang M H. Caspase-dependent Mcl-1 cleavage and effect of Mcl-1 phosphorylation in ABT-737-induced apoptosis in human acute lymphoblastic leukemia cell lines. Exp Biol Med Maywood NJ. 2014; 239:1390-402.
42. Tan S F, Pearson J M, Feith D J, Loughran T P. The emergence of acid ceramidase as a therapeutic target for acute myeloid leukemia. Expert Opin Ther Targets. 2017; 21:583-90.
43. Coant N, Sakamoto W, Mao C, Hannun Y A. Ceramidases, roles in sphingolipid metabolism and in health and disease. Adv Biol Regul. 2017; 63:122-31.
44. Morad S A F, Tan S-F, Feith D J, Kester M, Claxton D F, Loughran T P, et al. Modification of sphingolipid metabolism by tamoxifen and N-desmethyltamoxifen in acute myelogenous leukemia—Impact on enzyme activity and response to cytotoxics. Biochim Biophys Acta. 2015; 1851:919-28.
45. Powell J A, Lewis A C, Zhu W, Toubia J, Pitman M R, Wallington-Beddoe C T, et al. Targeting sphingosine kinase 1 induces MCL1-dependent cell death in acute myeloid leukemia. Blood. 2017; 129:771-82.
46. Lin C-F, Tsai C-C, Huang W-C, Wang Y-C, Tseng P-C, Tsai T-T, et al. Glycogen Synthase Kinase-3β and Caspase-2 Mediate Ceramide- and Etoposide-Induced Apoptosis by Regulating the Lysosomal-Mitochondrial Axis. Zhivotovsky B, editor. PLOS ONE. 2016; 11:e0145460.
47. Massiello A, Chalfant C E. SRp30a (ASF/SF2) regulates the alternative splicing of caspase-9 pre-mRNA and is required for ceramide-responsiveness. J Lipid Res. 2006; 47:892-7.
48. Thomas L W, Lam C, Edwards S W. Mcl-1; the molecular regulation of protein function. FEBS Lett. 2010; 584:2981-9.
49. Makishima H, Visconte V, Sakaguchi H, Jankowska A M, Abu Kar S, Jerez A, et al. Mutations in the spliceosome machinery, a novel and ubiquitous pathway in leukemogenesis. Blood. 2012; 119:3203-10.
50. Wang Q, Wan J, Zhang W, Hao S. MCL-1 or BCL-xL-dependent resistance to the BCL-2 antagonist (ABT-199) can be overcome by specific inhibitor as single agents and in combination with ABT-199 in acute myeloid leukemia cells. Leuk Lymphoma. 2019; 1-11.
51. Wei A, Strickland S A, Hou J-Z, Fiedler W, Lin T L, Walter R B, et al. Venetoclax with Low-Dose Cytarabine Induces Rapid, Deep, and Durable Responses in Previously Untreated Older Adults with AML Ineligible for Intensive Chemotherapy. Blood. 2018; 132:284-284.
52. DiNardo C D, Pratz K, Pullarkat V, Jonas B A, Arellano M, Becker P S, et al. Venetoclax combined with decitabine or azacitidine in treatment-naive, elderly patients with acute myeloid leukemia. Blood. 2019; 133:7-17.
53. Hengst J A, Dick T E, Sharma A, Doi K, Hegde S, Tan S F, Geffert L M, Fox T E, Sharma A K, Desai D, Amin S, Kester M, Loughran T P, Paulson R F, CLaxton D F, WAng H G, and Yun J K. A Multitargeted Inhibitor of Shingosine Kinase and Microtubule Dynamics Demonstrating Therapeutic Efficacy in Acute Myeloid Leukemia Models. Cancer Transl Med. 2017; 3:109-121.
54. Kester M, Bassler J, Fox T E, Carter C J, Davidson J A and Parette M R. Preclinical Development of a C6-ceramide NanoLlposome, a Novel Sphingolipid Therapeutic. Biol Chem. 2015; 396,737-737.
55. Hankins J L, Doshi U A, Haakenson J K, Young M M, Barth B M and Kester M. The Therapeutic Potential of Nanoscale Sphingolipid Technologies. Handb Exp Pharmaccol. 2013; 197-210.
56. McDaniel DK, Jo A, Ringel-Scaia V M, Coutermarsh-Ott S, Rothschild, D E, Powell, M D, Zhang R, Long T E, Oestreich K J, Riffle J S, Davis R M, and Allen I C. TIPS Pentacene Loaded PEO-PDLLA Core-Shell Nanoparticles Have Similar Cellular Uptake Dynamics in M1 and M2 Macrophages and in Corresponding in vivo Microenvironments. Nanomedicine. 2017; 13: 1255-1266.
57. Saad W S and Prud'homme R K. Principles of Nanoparticle Formation by Flash Nanaoprecipitation. Nano Today. 2016; 11:212-227.
58. Wei A H, Strickland S A Jr., Hou J Z, Fiedler W, Lin T L, Walter R B, Enjeti A, Tiong I S, Humerickhouse R, Hong W J, Hayslip J and Roboz G J. Venetoclax Combined with Low-Dose Cytarabine for Previously Untreated Patients with Acute Myeloid Leukemia: Results from a Phase Ib/II Study. J Clin Oncol. 2019; JC01801600, doi: 10.1200/JCO.18.01600.
59. DiNardo C D, Rausch C R, Benton C, Kadia T, Jain N, Pemmaraju N, Daver N, Covert W, Marx K R, Mace M, Jabbour E, Cortes J, Carcia-Manero G., Ravandi F., Bhalla K N, Kantarjian H, and Konopleva M. Clinical Experience with the BCL1-inhbitor Venetoclax in Combination Therapy for Relapsed and Refractory Acute Myeloid Leukemia and Related Myeloid Malignancies. Am J Hematol. 2018; 93:401-407.
60. Dombret, H.; Seymour, J. F.; Butrym, A.; Wierzbowska, A.; Selleslag, D.; Jang, J. H.; Kumar, R.; Cavenagh, J.; Schuh, A. C.; Candoni, A.; Recher, C.; Sandhu, I.; Bernal del Castillo, T.; Al-Ali, H. K.; Martinelli, G.; Falantes, J.; Noppeney, R.; Stone, R. M.; Minden, M. D.; McIntyre, H.; Songer, S.; Lucy, L. M.; Beach, C. L.; Dohner, H., International phase 3 study of azacitidine vs conventional care regimens in older patients with newly diagnosed AML with >30% blasts. Blood 2015, 126 (3), 291-9.
61. Huls, G.; Chitu, D. A.; Havelange, V.; Jongen-Lavrencic, M.; van de Loosdrecht, A. A.; Biemond, B. J.; Sinnige, H.; Hodossy, B.; Graux, C.; Kooy, R. V. M.; de Weerdt, O.; Breems, D.; Klein, S.; Kuball, J.; Deeren, D.; Terpstra, W.; Vekemans, M. C.; Ossenkoppele, G. J.; Vellenga, E.; Lowenberg, B.; Dutch-Belgian Hemato-Oncology Cooperative, G., Azacitidine maintenance after intensive chemotherapy improves DFS in older AML patients. Blood 2019, 133 (13), 1457-1464.
62. Aldoss, I.; Yang, D.; Aribi, A.; Ali, H.; Sandhu, K.; Al Malki, M. M.; Mei, M.; Salhotra, A.; Khaled, S.; Nakamura, R.; Snyder, D.; O'Donnell, M.; Stein, A. S.; Forman, S. J.; Marcucci, G.; Pullarkat, V., Efficacy of the combination of venetoclax and hypomethylating agents in relapsed/refractory acute myeloid leukemia. Haematologica 2018, 103 (9), e404-e407.
63. DiNardo, C. D.; Jonas, B. A.; Pullarkat, V.; Thirman, M. J.; Garcia, J. S.; Wei, A. H.; Konopleva, M.; Dohner, H.; Letai, A.; Fenaux, P.; Koller, E.; Havelange, V.; Leber, B.; Esteve, J.; Wang, J.; Pejsa, V.; Hajek, R.; Porkka, K.; Illes, A.; Lavie, D.; Lemoli, R. M.; Yamamoto, K.; Yoon, S. S.; Jang, J. H.; Yeh, S. P.; Turgut, M.; Hong, W. J.; Zhou, Y.; Potluri, J.; Pratz, K. W., Azacitidine and Venetoclax in Previously Untreated Acute Myeloid Leukemia. N Engl J Med 2020, 383 (7), 617-629.
64. Saied, E. M.; Arenz, C. Small Molecule Inhibitors of Ceramidases, Cell Physiol Biochem 2014, 34, 197-212.
65. Bai, A.; Szulc, Z. M.; Bielawski, J.; Pierce, J. S.; Rembiesa, B.; Terzieva, S.; Mao, C.; Xu, R.; Wu, B.; Clarke C. J.; Newcomb, B.; Liu, X.; Norris, J.; Hannun, Y. A.; Bielawska, A. Targeting (Cellular) Lysosomal Acid Ceramidase By B13: Design, Synthesis and Evaluation of Novel DMG:B13 Ester Prodrugs. Bioorg Med Chem 2014, 22, 6933-6944.
66. Realini, N.; Palese, F.; Pizzirani, D.; Pontis, S.; Basit, A.; Ganesan, A.; Piomelli, D. Acid Ceramidase in Melanoma: Expression, Localization and Effects of Pharmocological Inhibition. JBC 2016, 291, 2422-2434.
67. Ortega, J. A.; Arencibia, J. M.; La Sala, G.; Borgogno, M.; Bauer, I.; Bono L.; Bracia, C.; Armirotti, A.; Girotto, S.; Ganesan A.; De Vivo, M.; Pharmacophore Identification and Scaffold Exploration to Discover Novel, Potent, and Chemically Stable Inhibitors of Acid Ceramidase in Melanoma Cells, J Med Chem 2017, 60 (13), 5800-5815.
68. Di Martino, S.; Tardia, P; Cilibrasi, V.; Caputo, S.; Mazzonna, M.; Russo, D.; Penna, I.; Realini, N.; Marbaroli, N.; Migliore, M.; Pizzirani, D.; Ottonello, G.; Bertozzi, S. M.; Armirotti, A.; Nguyen, D.; Sun, Y.; Bongarzone, E. R.; Lansbury, P.; Liu, M.; Skerlj, R.; Scarpelli, R.; Lead Optimization of Benzoxazolone Carboxamides as Orally Bioavailable and CNS Penetrant Acid Ceramidase Inhibitors, J Med Chem 2020, 63, 3634-3664.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method for treating a disease, disorder, or condition associated with an acid ceramidase (AC) biological activity, the method comprising administering to a subject in need thereof a composition comprising: (a) an AC inhibitor; and (b) at least one additional active agent, wherein the at least one additional active agent comprises at least one of a ceramide nanoliposome (CNL), an inhibitor of a Bcl-2 family protein, a hypomethylating agent, cytarabine (AraC), daunorubicin, a Hedgehog pathway inhibitor, a FMS-like tyrosine kinase 3 (FTL3) inhibitor, an isocitrate dehydrogenase 1 and/or 2 (IDH1/2) inhibitor, and an antibody conjugate, wherein the composition is administered via a route and in an amount effective for reducing the AC biological activity, thereby treating and/or preventing the disease, disorder, or condition associated with the AC biological activity.

2. The method of claim 1, wherein the disease, disorder, or condition associated with the AC biological activity is a cancer.

3. The method of claim 1, wherein the cancer is acute myeloid leukemia (AML), optionally a venetoclax-resistant AML.

4. The method of claim 1, wherein the AC inhibitor is N-[(2S,3R)-1,3-dihydroxyoxtadecan-2-yl]-2-chloroacetamide (SACLAC) or a pharmaceutically acceptable salt thereof.

5. The method of claim 1, wherein the at least one additional active agent comprises a CNL, optionally a C6-CNL.

6. The method of claim 1, wherein the at least one additional active agent comprises a Bcl-2 inhibitor and/or a Mcl-1 inhibitor.

7. The method of claim 6, wherein the Bcl-2 inhibitor is venetoclax.

8. The method of claim 6, wherein the at least one additional active agent further comprises AraC and/or a hypomethylating agent, optionally wherein the hypomethylating agent is azacytidine (AZA).

9. The method of claim 1, wherein the at least one additional active agent comprises AraC, optionally wherein the at least one additional active agent comprises AraC in combination with one or more of venetoclax, daunorubicin, and a Hedgehog pathway inhibitor, optionally glasdegib.

10. The method of claim 1, wherein the at least one additional active agent comprises a hypomethylating agent, optionally wherein the hypomethylating agent is decitabine and/or azacytidine (AZA).

11. The method of claim 1, wherein the at least one additional active agent comprises an antibody conjugate, optionally gemtuzumab ozogamicin, and/or a Hedgehog pathway inhibitor, optionally glasdegib.

12. The method of claim 1, wherein the at least one additional active agent further comprises one or more of ivosidenib, enasidenib, midostaurin, gilteritinib, a histone deacetylase (HDAC) inhibitor, an epigenetic regulator, and a histone demethylase inhibitor.

13. A method for treating acute myeloid leukemia (AML), the method comprising administering to a subject in need thereof a composition comprising (a) an AC inhibitor; and (b) at least one additional active agent, wherein the at least one additional active agent comprises at least one of a ceramide nanoliposome (CNL), an inhibitor of a Bcl-2 family protein, a hypomethylating agent, cytarabine (AraC), daunorubicin, a Hedgehog pathway inhibitor, a FMS-like tyrosine kinase 3 (FTL3) inhibitor, an isocitrate dehydrogenase 1 and/or 2 (IDH1/2) inhibitor, and an antibody conjugate, wherein the composition is administered via a route and in an amount effective for treating the AML.

14. The method of claim 13, wherein the AC inhibitor is N-[(2S,3R)-1,3-dihydroxyoxtadecan-2-yl]-2-chloroacetamide (SACLAC) or a pharmaceutically acceptable salt thereof.

15. The method of claim 13, wherein the at least one additional active agent comprises a CNL, optionally a C6-CNL.

16. The method of claim 13, wherein the at least one additional active agent comprises a Bcl-2 inhibitor.

17. The method of claim 16, wherein the Bcl-2 inhibitor is venetoclax.

18. The method of claim 13, wherein the at least one additional active agent comprises a Mcl-1 inhibitor.

19. The method of claim 16, wherein the at least one additional active agent further comprises AraC and/or a hypomethylating agent, further optionally wherein the hypomethylating agent is azacytidine (AZA).

20. The method of claim 13, wherein the at least one additional active agent comprises AraC, optionally wherein the at least one additional active agent comprises AraC in combination with one or more of venetoclax, daunorubicin, and a Hedgehog pathway inhibitor, optionally glasdegib.

21. The method of claim 13, wherein the at least one additional active agent comprises a hypomethylating agent, optionally wherein the hypomethylating agent is decitabine and/or azacytidine (AZA).

22. The method of s an antibody conjugate, optionally gemtuzumab ozogamicin, and/or a Hedgehog pathway inhibitor, optionally glasdegib.

23. The method of claim 13, wherein the at least one additional active agent further comprises one or more of ivosidenib, enasidenib, midostaurin, gilteritinib, a histone deacetylase (HDAC) inhibitor, an epigenetic regulator, and a histone demethylase inhibitor.

24. The method of claim 13, wherein the AML is a venetoclax-resistant AML.

25. The method of claim 13, wherein the AC inhibitor and/or the one or more additional active agent is encapsulated in a polymer nanoparticle, wherein the polymer nanoparticle comprises a biodegradable and biocompatible polymer, optionally wherein the biodegradable and biocompatible polymer is selected from a polyethylene glycol-poly(lactic-co-glycolic acid) (PEG-PLGA) copolymer and a polyethylene glycol-poly(d,l-lactic acid) (PEG-PDLLA) diblock copolymer.

26. The method of claim 1, wherein the subject is a mammalian subject, optionally a human subject.

27. A composition for use in treating a disease, disorder, or condition associated with an acid ceramidase (AC) biological activity, the composition comprising (i) an AC inhibitor, optionally N-[(2S,3R)-1,3-dihydroxyoctadecan-2-yl]2-chloroacetamide (SACLAC) or a pharmaceutically acceptable salt thereof, and (ii) at least one additional active agent, wherein the at least one additional active agent comprises at least one of a ceramide nanoliposome (CNL), an inhibitor of a Bcl-2 family protein, a hypomethylating agent, cytarabine (AraC), daunorubicin, a Hedgehog pathway inhibitor, a FMS-like tyrosine kinase 3 (FTL3) inhibitor, an isocitrate dehydrogenase 1 and/or 2 (IDH1/2) inhibitor, and an antibody conjugate, in an amount effective for reducing the AC biological activity in a subject.

28. A composition for use in treating acute myeloid leukemia (AML), the composition comprising (i) an acid ceramidase (AC) inhibitor, optionally N-[(2S,3R)-1,3-dihydroxyoctadecan-2-yl]2-chloroacetamide (SACLAC) or a pharmaceutically acceptable salt thereof and (ii) at least one additional active agent, wherein the at least one additional active agent comprises at least one of a ceramide nanoliposome (CNL), an inhibitor of a Bcl-2 family protein, a hypomethylating agent, cytarabine (AraC), daunorubicin, a Hedgehog pathway inhibitor, a FMS-like tyrosine kinase 3 (FTL3) inhibitor, an isocitrate dehydrogenase 1 and/or 2 (IDH1/2) inhibitor, in an amount effective for treating AML in a subject.

29. The composition of claim 27, wherein the at least one additional active agent comprise at least one of a B-cell lymphoma 2 protein (Bcl-2) inhibitor, optionally venetoclax, a ceramide nanoliposome (CNL), optionally a C6-ceramide nanoliposome, and a hypomethylating agent, optionally decitabine and/or azacytidine (AZA).

30. The composition of claim 27, wherein the at least one additional active agent comprises ventoclax, AraC, and/or AZA.

31. The composition of claim 30, wherein the composition comprises SACLAC, AraC, and venetoclax.

32. The composition of claim 27, wherein the at least one additional active agent comprises daunorubicin, glasdegib, and/or gemtuzumab ozogamicin.

33. The composition of claim 27, further comprising one or more of ivosidenib, enasidenib, midostaurin, gilteritinib, a histone deacetylase (HDAC) inhibitor, an epigenetic regulator, and a histone demethylase inhibitor.

34. The composition of claim 27, wherein the AC inhibitor and/or one or more of the one or more additional active agents are encapsulated in a polymer nanoparticle, wherein the polymer nanoparticle comprises a biodegradable and biocompatible polymer, optionally wherein the biodegradable and biocompatible polymer is selected from a polyethylene glycol-poly(lactic-co-glycolic acid) (PEG-PLGA) copolymer and a polyethylene glycol-poly(d,l-lactic acid) (PEG-PDLLA) diblock copolymer.

35. A composition comprising an AC inhibitor, optionally SACLAC, encapsulated in a ceramide nanoliposome (CNL) or a polymer nanoparticle, wherein the polymer nanoparticle comprises a biodegradable and biocompatible polymer selected from a polyethylene glycol-poly(lactic-co-glycolic acid) (PEG-PLGA) copolymer and a polyethylene glycol-poly(d,l-lactic acid) (PEG-PDLLA) diblock copolymer.


SEQUENCE LISTING (2 sequences)

SEQ ID NO: 1 - DNA, 20 bp, Artificial sequence
  synthetic sequence
gaggaggacgagttgtaccg

SEQ ID NO: 2 - DNA, 20 bp, Artificial sequence
  synthetic sequence
actccacaaacccatccttg